(19) United States
(12) Patent Application Publication
(10) Pub. No.: US 2023/0181801 A1
(43) Pub. Date: Jun. 15, 2023

(54) MOLECULAR IODINE-INFUSED POLYMERS, ARTICLES, AND PRODUCTS, AND THEIR PREPARATION AND USE

(71) Applicant: I2PURE CORP., Reston, VA (US)

(72) Inventor: Jack KESSLER, Southborough, MA (US)

(21) Appl. No.: 18/064,239

(22) Filed: Dec. 9, 2022

Related U.S. Application Data
(60) Provisional application No. 63/265,195, filed on Dec. 9, 2021.
(60) Provisional application No. 63/288,487, filed on Dec. 10, 2021.
(60) Provisional application No. 63/268,128, filed on Feb. 16, 2022.

(51) Int. Cl. A61L 29/16; A61L 29/04

(52) U.S. Cl. CPC A61L 29/16; A61L 29/04; A61L 2300/404; A61L 2430/04

(57) ABSTRACT

Molecular iodine-infused catheters disclosed herein have biocidal effects and may be used to treat or prevent urinary tract infection (UTI). Other molecular iodine-infused polymers, articles, and products, and their preparation and uses are also disclosed.

### CROSS-REFERENCE TO RELATED APPLICATIONS

This application claims the benefit of U.S. Provisional Patent Application No. 63/265,195, filed Dec. 9, 2021; 63/288,487, filed Dec. 10, 2021; and 63/268,128, filed Feb. 16, 2022; all of which are incorporated by reference in their entireties herein. U.S. patent application Ser. No. 16/088,992, filed Jul. 19, 2017 and Ser. No. 17/402,467, filed Aug. 13, 2021, and International Application Nos. PCT/US2017/042726, filed Jul. 19, 2017 and PCT/US2021/046035, filed Aug. 13, 2021 are also incorporated by reference in their entireties herein.

### TECHNICAL FIELD

This invention relates to molecular iodine-infused polymers, articles, and products, and their preparation and use.

### BACKGROUND

Polymers are widely used in medical devices, e.g., catheters. Polymer based or associated infections remain a significant issue in healthcare. For example, about 40% of infections in healthcare are urinary tract infection (UTI), and 80% of UTI is triggered by catheter placement. More than 10% of the adult patients used indwelling catheters during a stay in the hospital, and 3% to 7% of patients had catheter-associated UTI. See, e.g., www.ncbi.nlm.nih.gov/pmc/articles/PMC6953942/#ref1.

In this application, we describe molecular iodine-infused polymers, articles and products, and their preparation and use, e.g., in prevention and treatment of infections.

### SUMMARY

### Overview

The present disclosure is directed to molecular iodine-infused polymers, molecular iodine-infused articles and molecular iodine-infused products, and their preparation and use.

Embodiments of the molecular iodine-infused polymers are disclosed. The molecular iodine-infused polymer comprises a polymer and molecular iodine, and releases molecular iodine. Molecular iodine is released from the molecular iodine-infused polymer. The molecular iodine-infused polymer is prepared by contacting the polymer with a molecular iodine preparation composition. In certain embodiments, the molecular iodine preparation composition is a high molecular iodine concentration composition disclosed herein. The composition may be a solution, a viscous solution, a cream, a lotion, a gel, an ointment, a spray, or a suspension.

Embodiments of the molecular iodine-infused articles are disclosed. In certain embodiments, the molecular iodine-infused article comprises one or more molecular iodine-infused polymers. In certain embodiments, the one or more molecular iodine-infused polymers form a coating of the article. Examples of the articles of molecular iodine-infused articles include, without limitation, catheters, sutures, grafts, stents, wound dressing material, bandage, artificial skin, implants, packaging to hold sterile medical materials, and polymers that provide a disinfecting atmosphere for materials (e.g., metals, polymers, fabrics, plants, and food) by virtue of being packaged with or placed adjacent to said materials.

Embodiments of the molecular iodine-infused products are disclosed. In certain embodiments, the molecular iodine-infused product comprises one or more molecular iodine-infused articles in one or more compartments, and may further include a molecular iodine storage composition. In certain embodiments, the molecular iodine-infused product may comprise additional compartments, the additional compartments may include a molecular iodine pre-treatment composition for pre-treating an article, such as an indwelling urinary catheter before use. When the compartment includes a molecular iodine pre-treatment composition, the product may include an article that may or may not be infused with molecular iodine. The additional compartments may comprise one or more un-infused articles, or a molecular iodine in-use composition for administration to a subject.

In certain embodiments, the molecular iodine in-use composition may be administered to the subject by being applied to the molecular-iodine infused article which is in contact with the subject or will be applied to the subject. For example, the in-use composition may be applied to the outer surface of the molecular-iodine infused article and applied to the subject when the molecular iodine-infused article contacts the subject. The in-use composition may be administered to the subject by delivering through the molecular iodine infused article. The molecular iodine-infused article may be prepared by contacting an un-infused polymeric article with a molecular iodine preparation composition. In certain embodiments, the molecular iodine storage composition, the pre-treatment composition, the molecular iodine in-use composition, and the molecular iodine preparation composition are embodiments of high molecular iodine concentration compositions disclosed herein. In certain embodiments, the molecular iodine storage composition, the pre-treatment composition, the in-use composition, and the preparation composition are the same. In other embodiments, the molecular iodine storage composition, the pre-treatment composition, the in-use composition, and the preparation composition are different. In some instances, the molecular iodine storage composition can be the same as the preparation composition but in other instances, the preparation composition can contain a higher or lower concentration of molecular iodine. In certain embodiments the articles may be treated with an in-use composition that is contacted to the article after it has been used for a time period. As an example, an indwelling urinary catheter may be initially treated with a preparation composition that contains a very high concentration of molecular iodine and placed in a storage composition that contains a higher or lower concentration of molecular iodine and then an in-use composition with a lower concentration of molecular iodine may be transferred into the interior of said catheter several days after the catheter has been placed in a subject, optionally, the catheter may be bathed in the pre-treatment composition after it is removed from the package and before it is placed in the subject.

In certain embodiments, the molecular iodine-infused product is a sanitizing chamber comprising a first compartment made of one or more molecular iodine-infused polymers. Molecular iodine released from the molecular iodine-infused polymers provides a disinfecting atmosphere for materials (e.g., metals, polymers, fabrics, plants, and food) may disinfect an article that is placed in the sanitizing chamber. In certain embodiments, the first compartment further includes one or more molecular iodine-infused polymers or articles therein. In certain embodiments, the first compartment includes one or more molecular iodine-infused polymers or articles therein but the first compartment itself is not made of a molecular iodine-infused polymer. In certain embodiments, the sanitizing chamber further comprises a second compartment separated from the first compartment by and a physical barrier. The barrier can be breakable to allow fluid communication between the first and second compartments.

### Biostatic Persistent, Biocidal Persistent, Prolonged Biocidal Activity, and Iodine Flux Rate

Molecular iodine is released from the molecular iodine-infused polymer or article. In certain embodiments, the molecular iodine-infused polymer or article is biostatic persistent. In certain embodiments, the molecular iodine-infused polymer or article is biocidal persistent. In certain embodiments, the molecular iodine-infused polymer or article has prolonged biocidal activity.

In certain embodiments, the molecular iodine-infused polymer or article has at least a 0.45 log, 0.776 log, 1 log, 1.639 log, 2 log, 3 log, 3.06 log, 3.14 log, 3.23 log, 3.39 log, 3.55 log, 4 log, 4.471 log, 5 log, 5.833 log, or 6 log biocidal persistent activity.

In certain embodiments, the molecular iodine-infused polymer or article has at least a 0.45 log, 0.776 log, 1 log, 1.639 log, 2 log, 3 log, 3.06 log, 3.14 log, 3.23 log, 3.39 log, 3.55 log, 4 log, 4.471 log, 5 log, 5.833 log, or 6 log prolonged biocidal activity.

In certain embodiments, the molecular iodine-infused polymer or article has an iodine flux rate measured by the mass of molecular iodine released per unit time per unit surface area or by the mass of molecular iodine released per unit time per unit mass of the molecular iodine-infused polymer or article. In certain embodiments, the iodine flux rate of an embodiment of the molecular iodine-infused polymer or article may be measured, e.g., by a method shown in Example 3B. In certain embodiments, the molecular iodine-infused polymer or article has an iodine flux rate sufficient to afford biostatic persistent, biocidal persistent, and prolonged biocidal activity.

In certain embodiments, the iodine flux rate of the molecular iodine-infused polymer or article drops about 90%, about 85%, about 80%, about 75%, about 70%, about 65%, about 60%, about 55%, about 50%, about 10%, and about 1.0%, after a time period.

### Molecular Iodine Concentrations of the Molecular Iodine-Infused Polymer or Article and Stability

In certain embodiments, the molecular iodine-infused polymer or article has a molecular iodine mass concentration characterized by the mass of molecular iodine per unit mass of the molecular iodine-infused polymer or article. Examples of the molecular iodine mass concentration includes, without limitation, at least about 13.0 mg/g, 5.16 mg/g, 4.54 mg/g. 2.32 mg/g, and ranges therebetween. See, e.g., Example 3A.

In certain embodiments, the molecular iodine-infused polymer or article has a molecular iodine volume concentration characterized by the mass of molecular iodine per unit volume of the molecular iodine-infused polymer or article.

### The High Molecular Iodine Concentration Compositions

In certain embodiments, the high molecular iodine concentration composition comprises molecular iodine at a concentration of about 100 ppm to about 1,120 ppm, about 300 ppm to about 1,274 ppm, about 1,274 ppm to about 170,000 ppm, about 1,274 ppm to about 160,000 ppm, about 1,274 ppm to about 150,000 ppm, about 1,274 ppm to about 100,000 ppm, about 1,274 ppm to about 66,000 ppm, about 1,274 ppm to about 66,000 ppm, about 1,274 ppm to about 15,200 ppm, about 1,400 ppm to about 68,000 ppm, about 1,400 ppm to about 15,200 ppm, about 1,400 ppm to about 66,000 ppm, or about 1,500 ppm by weight/volume (wt/v); and an organic carrier having a concentration of no less than about 93.5% wt, no less than about 95% wt, no less than about 98% wt, or no less than about 99% wt of the total weight of the high molecular iodine concentration composition.

In certain embodiments, the organic carrier of the high molecular iodine concentration composition is a pharmaceutically acceptable organic carrier, and the high molecular iodine concentration composition is a pharmaceutical formulation. In certain embodiments, the high molecular iodine concentration pharmaceutical formulation further comprises a second pharmaceutically acceptable carrier and may contain other ingredients to improve stability or impart other characteristics to the product (e.g. lubricity).

### Uses

Embodiments of a method of treating or preventing a condition associated with a microorganism in a subject comprising contacting the subject with one or more molecular iodine-infused polymers or articles disclosed herein. In certain embodiments, the microorganism is killed at the contact site or in vicinity of the contact site. In certain embodiments, the growth rate of the microorganism is reduced at the contact site or in vicinity of the contact site. In certain embodiments, the method further comprises delivering a therapeutically effective amount or prophylactically effective amount of molecular iodine, high molecular iodine concentration composition, or high molecular iodine concentration pharmaceutical formulation via the molecular iodine infused polymers or articles. In certain embodiments, the microorganism is present at the contact site or in vicinity of the contact site in or on the subject. In certain embodiments, a biofilm is present at the contact site or vicinity of the contact site. In certain embodiments, more than one species of microorganism are present at the contact site or vicinity of the contact site. In certain embodiments, the biofilm comprises more than one species of microorganism, e.g., 2, 3, 4, 5, or 6 species of microorganism (See Example 2(A)).

Examples of the conditions treatable or preventable include, without limitation, infections caused by microorganism, e.g., urinary tract infection, breast implant infections, and wound infection. In certain embodiments, the infection is chronic infection. In certain embodiments, the infection contains microorganisms that are resistant to antibiotics. In certain embodiments, the infection is a chronic urinary tract infection.

Provided herein are embodiments of method of fostering wound-healing or preventing scar of a wound of a subject comprising contacting the wound with one or more of the molecular iodine-infused polymers or molecular iodine-infused articles (e.g., wound dressing and artificial skin) or molecular iodine-infused products.

Provided herein are embodiments of a method of treating a surgical site to foster healing, or prevent infection in a subject in need comprising contacting the subject with the molecular iodine-infused polymer or molecular iodine-infused article (e.g., wound dressing and artificial skin), e.g., at the surgical site. In certain embodiments, the method of treating a surgical site is performed as a pre-surgical treatment, a post-surgical treatment, or a treatment during a surgery. Provided herein are embodiments of sanitizing one or more articles or materials (e.g., metals, polymers, fabrics, plants, and food) comprising placing the one or more articles or materials adjacent to one or more molecular iodine-infused polymers or one or more molecular iodine-infused articles. In certain embodiments, the one or more articles or materials are placed in a sealed compartment comprising the one or more molecular iodine-infused polymers or one or more molecular iodine-infused articles. In certain embodiments, the sealed compartment is made of the one or more molecular iodine-infused polymers. In certain embodiments, the sealed compartment comprises one or more molecular iodine-infused polymers or articles placed therein.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

FIGS. 1A-1H: Biocidal effects of embodiments of molecular iodine-infused catheters prepared by treatment with various molecular iodine preparation compositions in glycerin. FIG. 1A: I₂-glycerin (14,670 ppm) treated catheter. FIG. 1B: I₂-glycerin (7,007 ppm) treated catheter. FIG. 1C: I₂-glycerin (3,376 ppm) treated catheter. FIG. 1D: I₂-glycerin (1,019 ppm) treated catheter. FIG. 1E: I₂-glycerin (587 ppm) treated catheter. FIG. 1F: I₂-glycerin (387 ppm) treated catheter. FIG. 1G: Glycerin treated catheter (negative control). FIG. 1H: Bacterial growth without any catheter (blank control).

FIGS. 2A-2F. Biocidal effects of embodiments of molecular iodine-infused catheters prepared by treatment with various molecular iodine preparation compositions in glycerin. FIG. 2A: I₂-glycerin (738 ppm) treated catheter. FIG. 2B: I₂-glycerin (301 ppm) treated catheter. FIG. 2C: I₂-glycerin (136 ppm) treated catheter. FIG. 2D: I₂-glycerin (25 ppm) treated catheter. FIG. 2E: Glycerin treated catheter (negative control). FIG. 2F: Bacterial growth without any catheter (blank).

FIGS. 3A-3L: Biocidal effects of embodiments of molecular iodine-infused catheters prepared by treatment with various molecular iodine preparation compositions in glycerin. FIG. 3A: I₂-glycerin (12,560 ppm) treated catheter. FIG. 3B: I₂-glycerin (10,100ppm) treated catheter. FIG. 3C: I₂-glycerin (7,961 ppm) treated catheter. FIG. 3D: I₂-glycerin (5,831ppm) treated catheter. FIG. 3E: I₂-glycerin (4,161 ppm) treated catheter. FIG. 3F: I₂-glycerin (3,688 ppm) treated catheter. FIG. 3G: I₂-glycerin (3,305 ppm) treated catheter. FIG. 3H: I₂-glycerin (3,196 ppm) treated catheter. FIG. 3I: I₂-glycerin (2,858 ppm) treated catheter. FIG. 3J: I₂-glycerin (2,025 ppm) treated catheter. FIG. 3K: I₂-glycerin (998 ppm) treated catheter. FIG. 3L: I₂-glycerin (730 ppm) treated catheter.

FIG. 4. I₂ flux versus I₂ concentration in molecular iodine preparation compositions. Square: 1/25″ thick Laimeisi rubber high temperature material (RSH-1/25″ silicone); circle: 1/32″ thick Laimeisi rubber high temperature material (RSH-1/32″ silicone); and triangle: 1/32″ thick latex sheets from MSC Industrial Company (MSC-1/32″ latex).

FIGS. 5A-5H: Comparison of biocidal effects of embodiments of molecular iodine-infused silicone prepared by treatment with I₂-glycerin and silicone treated with aqueous iodine solution. FIG. 5A: Representative images of Candida albicans ATCC® 90028TM treated with I₂-glycerin (top row) or aqueous Iodine (bottom row) at 24 h and 48 h. FIG. 5B: Representative images of Enterococcus faecalis ATCC 51299 treated with I₂-glycerin (top row) or aqueous Iodine (bottom row) at 24 h and 48 h. FIG. 5C: Representative images of Enterococcus faecium ATCC 700221 treated with I₂-glycerin (top row) or aqueous Iodine (bottom row) at 24 h and 48 h. FIG. 5D: Representative images of Klebsiella pneumoniae ATCC 700603 Enterococcus faecium ATCC 700221 treated with I₂-glycerin (top row) or aqueous Iodine (bottom row) at 24 h; and 48 h. FIG. 5E: Representative images of Pseudomonas aeruginosa ATCC 15442 treated with I₂-glycerin (top row) or aqueous Iodine (bottom row) at 24 h and 48 h. FIG. 5F: Representative images of Proteus mirabilis ATCC 29245 treated with I₂-glycerin (top row) or aqueous Iodine (bottom row) at 24 h and 48 h. FIG. 5G: Representative images of Staphylococcus aureus ATCC 6538 treated with I₂-glycerin (top row) or aqueous Iodine (bottom row) at 24 h and 48 h. FIG. 5H: Representative images of Staphylococcus epidermidis ATCC 51625 treated with I₂-glycerin (top row) or aqueous Iodine (bottom row) at 24 h and 48 h.

FIGS. 6A-6F: I₂ flux of RSH-1/32″ treated with various aqueous iodine solutions. FIG. 6A: I₂ flux of RSH-1/32″ treated with Lugol's Iodine Solution. FIG. 6B: I₂ flux of RSH-1/32″ treated with Iodine Tincture. FIG. 6C: I₂ flux of RSH-1/32″ treated with Iodine Solution. FIG. 6D: I₂ flux of RSH-1/32″ treated with Povidone-I₂ solution. FIG. 6E: I₂ flux of RSH-1/32″ treated with aqueous iodine solution. FIG. 6F: I₂ flux of RSH-1/32″ treated with aqueous iodine solution with methanol (−1% by wt).

FIG. 7: I₂ flux for I₂infused Bardia silicone catheter (circle) and Hollister latex catheter (square) treated with I₂-propylene glycol (17,000 ppm).

FIGS. 8A-8G: Images of embodiments of molecular iodine-infused articles and polymers after left in ambient environment. FIG. 8A: Image of molecular iodine-infused silicone breast implant treated with 17,000 ppm I₂-glycerin, after left in ambient environment. FIG. 8B: Time 0 hours: Bardia Foley Catheter Material (A); Amsure Silicone Foley Catheter (B); Cold and Colder, Ultra Clear Platinum Silicone Tubing (C); and FJYQOP Silicone Nipple Covers (D); I₂ concentration of molecular iodine preparation composition I₂-propylene glycol: 5,000 ppm (row 1); 10,000 ppm (row 2); 20,000 ppm (row 3); and 40,000 ppm (row 4); and 80,000 ppm (row 5). FIG. 8C: Time 37.5 hours after the embodiments of molecular iodine-infused articles and polymers of FIG. 8B left in ambient environment. FIG. 8D: Time 208 hours. after the embodiments of molecular iodine-infused articles and polymers of FIG. 8B left in ambient environment. FIG. 8E: Bardia Foley catheter material saturated with 10,000 ppm I₂-glycerin and then washed in distilled water and left in ambient environment at 0, 2, 26.5 54, 95.5 and 250.75 hours. FIG. 8F: Bardia Foley catheter material saturated with 10,000 ppm I₂-propylene glycol and then washed in distilled water and left in ambient environment at 0, 4.5, 54.6, 59.25, 68 and 83.75 hours. FIG. 8G: Amsure silicone Foley catheter material saturated with 10,000 ppm I₂-glycerin and then washed in distilled water and left in ambient environment at 0, 0.5, 0.75 9.5, 31 and 69.5 hours.

### DETAILED DESCRIPTION

The molecular iodine-infused polymers, articles, and products disclosed herein are especially useful for treating or preventing infection when the articles or polymers need to be kept in a subject for days, weeks, months, or longer. For example, about 40% of infections in healthcare are urinary tract infection (UTI), and 80% of UTI is triggered by catheter placement. More than 10% of the adult patients used indwelling catheters during a stay in the hospital, and 3% to 7% of patients had catheter-associated UTI. See, e.g., www.ncbi.nlm.nih.gov/pmc/articles/PMC6953942/#ref1. The Examples section provides embodiments of the molecular iodine-infused polymers and articles that provide an iodine flux with biocidal effects for days, weeks, and over 30 days. An embodiment of molecular iodine-infused catheter showed biocidal effects against biofilms having one or five common microorganism species in UTI (Example 2A), e.g., at least 3 log reduction against a biofilm developed with five most common microorganisms related to UTI: S. aureus (3.06 log reduction), P. aeruginosa (3.14 log reduction), E. coli (3.39 log reduction), E. faecalis (3.23 log reduction), and C. freundii (3.55 log reduction). Embodiments of molecular iodine-infused catheter material of silicone (Shenzhen Laimeisi Silicone Industry Co., Ltd., Shenzhen, China, hereinafter RSH, 1.016 mm thick) were also prepared and showed biocidal effects against species of a biofilm developed with P. aeruginosa (5.833 log reduction) and S. aureus (4.471 log reduction) (Example 2A). Thus, molecular iodine-infused catheters disclosed herein may be used to prevent or treat UTI. Biocidal effects measured under dynamic contact conditions by ASTM Method E2149-20 also showed a>4 log kill of bacteria of molecular iodine-infused silicone catheters against E. coli and S. aureus, respectively (Example 2B). Various embodiments of molecular iodine-infused catheters were prepared by contacting samples of catheters with different materials (e.g., silicone and latex) or silicone or latex materials for catheters with various molecular iodine preparation compositions (e.g., Examples 2-5) having molecular iodine concentrations from 25 ppm to 100,000 ppm. The molecular iodine-infused catheters turned from clear to dark brown after exposure to the high molecular iodine concentration compositions and their color faded to clear or no further changes after about 12 hours, 19 hours, about 65 hours, about 37.5 hours, about 69.5 hours, about 83.75 hours, about 208 hours, or about 250.75 hours (Example 4) which reflected the outgassing of the molecular iodine because the molecular iodine was absorbed and outgassed by the catheter material. Biocidal effects (e.g., biostatic persistence, biocidal persistence, and prolonged biocidal activity) of embodiments of molecular iodine-infused catheters and catheter materials were observed when the molecular iodine-infused polymers or articles were exposed to various microorganism challenges (Example 2). Examples of microorganism challenges tested include Candida albicans, C. freundii, Escherichia coli, E. faecalis, Enterococcus faecium, Klebsiella pnuemoniae, Proteus mirabilis, Pseudomonas aeruginosa, Staphylococcus aureus, or Staphylococcus epidermidis streaked on suitable agar plates such as MacConkey agar plates, or presented in buffer or in biofilms (Example 2). Iodine flux rates sufficient to provide biocidal activity, biostatic persistence, biocidal persistence, or prolonged biocidal activity were observed and measured. Molecular iodine-infused polymers and articles were further characterized by molecular iodine concentrations of the polymers or articles by mass or by volume (Example 3A) or iodine flux rates (Example 3B).

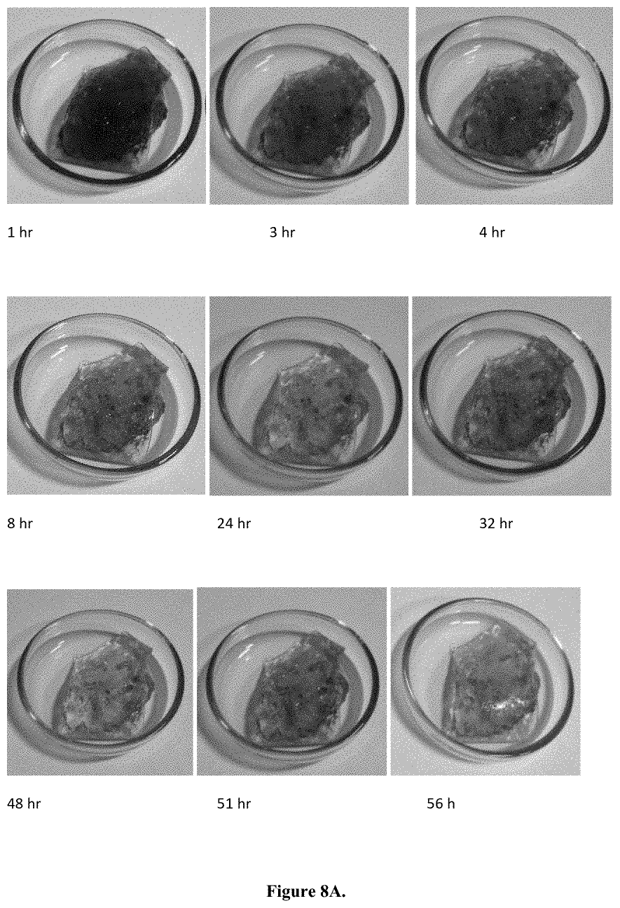

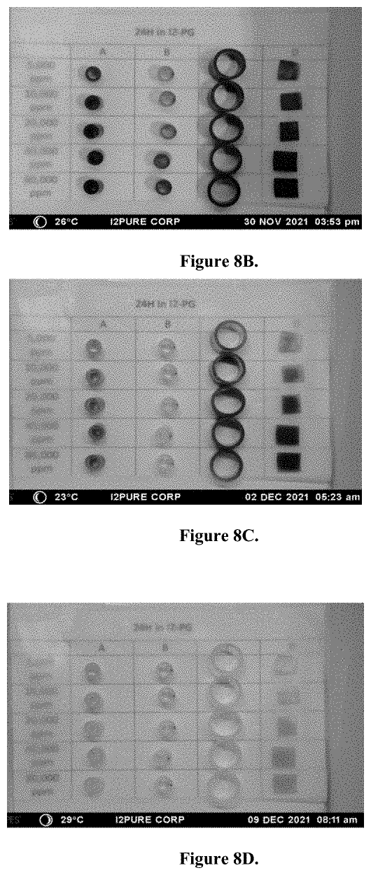

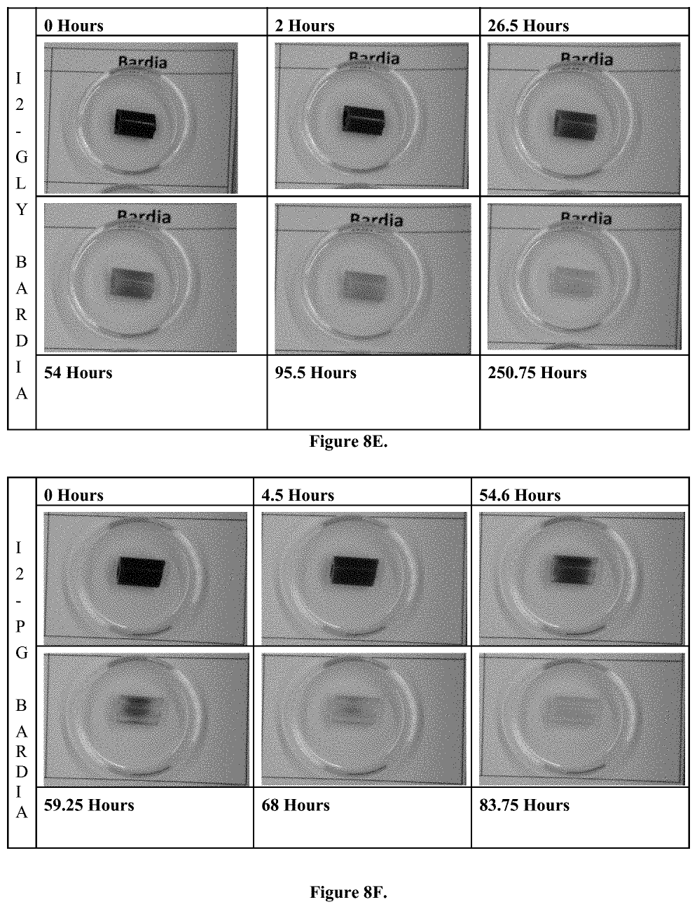

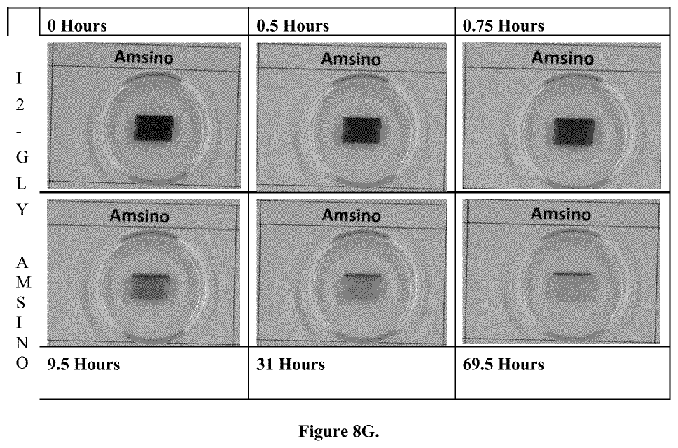

Breast implant infections are usually caused by Staphylococcus aureus and coagulase-negative staphylococci. See, e.g., www.ijidonline.com/article/S1201-9712(15)00096-X/fulltext#:˜:text=Breast%20implant%20infections%20are%20usually,aureus%20and%20coagulase%2Dnegative%20staphylococci. An embodiment of molecular iodine-infused breast silicone implant (Natrelle Inspira, 340 cc; style SRX; Lot 3089145 from Allergan, Irvine Calif.) was prepared by contacting with 17 k ppm I₂ in glycerin for 90 hrs. Regions of the implant turned dark brown after exposure to the high molecular iodine concentration composition and the color faded away, turned dark again (32 hr), faded, turned darker again (51 hr) and faded (Example 4, FIG. 8A), suggesting molecular iodine-infused breast implant released molecular iodine for at least two days and may be used to prevent or reduce infection in breast implant procedures. This particular breast implant was prepared from a liquid injection molding process.

The two most common bacteria that cause skin infections are Group A streptococcus (GAS) and Staphylococcus aureus. See, e.g., www.health.ny.gov/diseases/communicable/athletic_skin_infections/bacterial.htm#:˜:text=What%20are%20the%20two%20most,%2C%20commonly%20called%20%22staph.%22. Embodiments of molecular iodine-infused wound pad material (Sentrex BioSponge™ obtained from Binova, which was a porous chitosan wound dressing) were prepared by contacting with 100 k ppm I₂ in propylene glycol, 10 k ppm I₂ in propylene glycol, 1 k ppm I₂ in propylene glycol, and 100 ppm I₂ in propylene glycol, respectively, for 30 minutes. The treated wound pad material was then placed on top of agar with actively growing bacteria (e.g., E. Coli) overnight, and no bacteria colonies were observed underneath the molecular iodine-infused wound pad material (Example 2D(i)). Another embodiment of molecular iodine-infused wound pad material was treated with I₂ in propylene glycol at concentrations of 500 ppm, 1 k ppm, 1.5 k ppm, 3 k ppm, 6 k ppm, and 9 k ppm, respectively, and then placed on top of agar with actively growing bacteria Pseudomonas aeruginosa or Staph aureus, respectively for 24 hours. Similarly, no bacteria colonies were observed underneath the molecular iodine-infused wound pad material after 24 hours (Example 2D(ii)).

Additional polymers and articles were treated with various molecular iodine preparation compositions and showed biocidal effects. Several materials (e.g., LLDPE, PTFE, and PET) were also tested for molecular iodine infusion but turned out to be substantially dormant to molecular iodine infusion and may be used as outer packaging of molecular iodine-infused polymers or articles.

Additional embodiments of molecular iodine-infused polymers and articles were prepared by contacting polymers or articles with molecular iodine preparation compositions in various organic carriers. Glycerin was more effective than propylene glycol in infusing molecular iodine into silicone samples tested, while propylene glycol was more effective than glycerin in infusing molecular iodine into latex samples tested. Both organic carriers are excellent lubricants for urinary catheters. Consequently, a molecular iodine-infused catheter or a catheter bathed in a pre-treatment modular iodine solution may require no further lubrication of insertion. See, e.g., Example 5.

### Definitions

The singular forms “a,” “an,” and “the” include plural referents unless the context clearly dictates otherwise. Thus, for example, reference to a “gelling agent” refers to a single gelling agent as well as to several different gelling agents, reference to an “excipient” includes a single excipient as well as two or more different excipients, and the like.

The term “comprising,” which is inclusive or open-ended and does not exclude additional unrecited elements or method steps, is intended to encompass as alternative embodiments, the phrases “consisting essentially of” and “consisting of” where “consisting of” excludes any element or step not specified and “consisting essentially of” permits the inclusion of additional unrecited elements or steps that do not materially affect the essential or basic and novel characteristics of the composition or method under consideration.

The term “optional” or “optionally” means that the subsequently described circumstance may or may not occur, so that the description includes instances where the circumstance occurs and instances where it does not.

The term “pharmaceutically acceptable” in reference to an entity or ingredient is one that causes no significant adverse toxicological effects in a subject when administered to the subject.

The term “silicone” refers to a silicone or silicone rubber. In certain embodiments, the silicone is medical grade silicone. In certain embodiments, silicone comprises a polysiloxane having a chemical formula [R₁(R₂)SiO]ₙ, where R₁ and R₂ are independently selected from the group consisting of substituted and unsubstituted alkyl (e.g., C₁-C₂₀, e.g., methyl, ethyl), substituted and unsubstituted aryl (e.g., phenyl), substituted and unsubstituted alkylaryl, and substituted and unsubstituted arylalkyl. The silicone (e.g., silicone rubber) can be free of or substantially free of other resins or polymers. Pure silicone (e.g., silicone rubber) refers to commercially available silicones (e.g., silicone rubbers) that do not include other monomers or polymers or that include only trace or incidental amounts of other monomers or polymers.

The term “molecular iodine” refers to diatomic iodine, which is represented by the chemical symbol I₂ (CAS Registry Number: 7553-56-2) whether dissolved, suspended or in a solid state. The term “molecular iodine” may also be referred to as “elemental iodine” when in the solid state and is sometimes represented as “I₂” in this application. The term “molecular iodine” may also be referred as “I_{2,}” “free molecular iodine,” “unbound molecular iodine,” “uncomplexed molecular iodine,” and “un-complexed molecular iodine” in the art. Chemical activity of molecular iodine is not reduced by association with or complexation with other polymers or iodide and therefore exhibits antimicrobial activity. In aqueous solutions only hypoiodious acid (HOT) and molecular iodine are biocidal. The active biocide in acidic iodine-based biocides is believed to be free molecular iodine.

The term “iodine flux” or “iodine flux rate” refers to the release of molecular iodine from a polymer. Quantitative measurements of the iodine flux are provided in units of the mass (e.g., μg I₂) of molecular iodine released from a polymer per unit time (e.g., seconds) per unit area of the polymer (e.g., cm²) or unit mass (e.g., grams).

The term “iodide” or “iodide anion” refers to the species which is represented by the chemical symbol I⁻ (CAS Registry Number: 20461-54-5). Suitable counter-ions for the iodide anion include sodium, potassium, calcium, and the like.

The term “all iodine species” in a sample refers to all iodine containing components in the sample.

The term “ratio of molecular iodine to all iodine species” in a composition refers to the iodine content of molecular iodine (I₂) in the composition divided by the iodine content of all iodine species in the composition.

The term “organic carrier” refers to an organic molecule in which molecular iodine can be dispersed and the organic molecule does not react with molecular iodine. Examples of organic carrier include glycols with molecular weight below 300 (e.g., propylene glycol, di-propylene glycol, glycerin), propylene glycol monomethyl ether acetate, dimethyl sulfoxide (DMSO), alcohols (e.g., ethanol, propanols such as isopropanol and 1-propanol), and any mixtures of the foregoing.

The term “gelling agent” or “viscosity enhancer” refers to an organic molecule that increases the viscosity of a composition. Examples of gelling agent or viscosity enhancer include hydroxypropyl methylcellulose (HPMC) and crosslinked polyacrylic acid polymers (e.g., Carbopols). Examples of the gelling agents for use in the high molecular iodine concentration compositions or pharmaceutical formulations may also include, without limitation, synthetic hydrocolloids like homopolymers of acrylic acid such as those offered by Lubrizol Advanced Materials, Inc., Cleveland, Ohio, including Ultrez 10®, Ultrez 20®, Ultrez 30® and the Carbopols including Carbopol® 934, Carbopol® 940, Carbopol® 980, Carbopol® SC-200; methyl glucoside derivatives; alcohol esters such as monohydric alcohol esters, polyhydric alcohol esters; polyethylene glycols (PEG) such as PEG-diisostearate, propoxylated PEG monolaurate, polyglyeryl-3-laurate, natural hydrocolloids like carrageenan, locust bean gum, guar gum, acacia, tragacanth, alginic acid, gelatin, and semisynthetic hydrocolloids, e.g., carboxymethyl cellulose, methyl cellulose and hydroxypropyl methyl cellulose. Examples of the viscosity enhancing agents for use in the high molecular iodine concentration compositions or pharmaceutical formulations may also include, without limitation, methyl cellulose, microcrystalline cellulose, carboxymethyl cellulose, hydroxypropyl cellulose, hydroxymethyl cellulose, hydroxyethyl cellulose, hydroxypropylmethyl cellulose, poloxamer (copolymers of polyoxypropylene and polyoxyethylene), cross-linked homopolymers of acrylic acid like Ultrez 30, and guar gum.

The term “stable” means that the variation of the molecular iodine content of the molecular iodine-infused polymers, articles, or products, or the high molecular iodine concentration composition or pharmaceutical formulation is less than 10% of the initial molecular iodine content.

The term “shelf-life” means the time period that the molecular iodine-infused polymers, articles, or products, the high molecular iodine concentration composition or pharmaceutical formulation remains stable in a package under a storage condition. In certain embodiments, the shelf-life is at least 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, or 5 years.

The term “effective amount” means an amount of the high molecular iodine concentration composition or pharmaceutical formulation that is needed to effectuate a desired clinical outcome from a subject the high molecular iodine concentration composition or pharmaceutical formulation is administered to.

The term “biostatic persistence” means that after the subject's contact with the molecular iodine-infused polymer or article or the high molecular iodine concentration composition or pharmaceutical formulation, the microorganism count at the contact site will be equal to or lower than baseline for a first time period post contact. The term “baseline” means the microorganism count at the site of contact immediately before contact of the molecular iodine-infused polymer or article or the high molecular iodine concentration composition or pharmaceutical formulation.

Unless otherwise specified, the term “biocidal persistence” means that at the subject's contact site of the molecular iodine-infused polymer or article or the high molecular iodine concentration composition or pharmaceutical formulation, wherein the site is subjected to a first microorganism challenge, the microorganism count reduction after a second time period post contact is at least about 1 log lower than the microorganism count at a site of the same microorganism challenge without the contact. The term “biocidal persistence” may be modified by the extent of microorganism reduction and the second time period the “biocidal persistence” lasts. For example, a 3 log/6 hours biocidal persistence means that at the site of the contact of the molecular iodine-infused polymer or article or the high molecular iodine concentration composition or pharmaceutical formulation the microorganism count measured at six hours post contact is reduced by about 3 log compared to the microorganism count at a site of the same microorganism challenge without the contact six hours post challenge. For example, a 2 log/12 hour biocidal persistence means that at the site of the contact of the molecular iodine-infused polymer or article or the high molecular iodine concentration composition or pharmaceutical formulation the microorganism count measured twelve hours post contact is reduced by about 2 log lower compared to the microorganism count at a site of the same microorganism challenge without the contact 12 hours later.

Unless otherwise specified, the term “prolonged biocidal activity” means that at the site of the contact of the molecular iodine-infused polymer or article or the high molecular iodine concentration composition or pharmaceutical formulation, wherein the site is subject to the first microorganism challenge and at least a second microorganism challenge during a third time period post contact, the microorganism count measured at the end of the third time period post contact is reduced at least about 1 log compared to the microorganism count at a site subjected to the same microorganism challenges at the end of the third time period. Similarly, the term prolonged biocidal activity may be further defined by the extent of microorganism reduction and the third time period the “prolonged biocidal activity” lasts. For example, a 3 log/6 hours prolonged biocidal activity means that at the site of the contact of molecular iodine-infused polymer or article or the high molecular iodine concentration composition or pharmaceutical formulation, the microorganism count measured six hours post contact is reduced about 3 log compared to the microorganism count at a site of the same microorganism challenges after 6 hours without the contact, wherein other than the first microorganism challenge, at least a second microorganism challenge is imposed within 6 hours post contact. For example, a 2 log/12 hour prolonged biocidal activity means that at the site of the contact of molecular iodine-infused polymer or article or the high molecular iodine concentration composition or pharmaceutical formulation, the microorganism count measured 12 hours post contact is reduced about 2 log compared to the microorganism count at a site of the same microorganism challenges after 12 hours without the contact, wherein other than the first microorganism challenge, at least a second microorganism challenge is imposed within 12 hours post contact.

The term “non-aqueous” means the total water content of the high molecular iodine concentration composition or pharmaceutical formulation is no more than 1.5% wt of the total weight of the composition.

The term “substantially non-aqueous” means the total water content of the high molecular iodine concentration composition or pharmaceutical formulation is no more than 5.0% wt of the total weight of the composition.

The term “water-free” means the total water content of the high molecular iodine concentration composition or pharmaceutical formulation is no more than 0.5% wt of the total weight of the composition.

The term “substantially water-free” means the total water content of the high molecular iodine concentration composition or pharmaceutical formulation is no more than 2.5% wt of the total weight of the composition.

The term “alcohol-free” means the total alcohol content of the high molecular iodine concentration composition or pharmaceutical formulation is no more than 0.75% wt of the total weight of the composition.

The term “substantially alcohol-free” means the total alcohol content of the high molecular iodine concentration composition or pharmaceutical formulation is no more than 5.5% wt of the total weight of the composition.

The term “PPM” of “ppm” of an agent in a composition means a weight/volume (wt/v) concentration of the agent in the composition expressed in parts per million, which may also be referred to as a mass/volume (m/v) concentration.

The term “surgical site” means an incision site on a subject, or any part of a subject's anatomy in organs or spaces which is opened or manipulated during a surgery.

The term “about” before a numeric value means a range of ±10% of the numeric value.

The present disclosure is directed to molecular iodine-infused polymers, molecular iodine-infused articles, and molecular iodine-infused products, and their preparation and use.

### I. Molecular Iodine-Infused Polymers

Embodiments of the molecular iodine-infused polymer are disclosed herein. The molecular iodine-infused polymer comprises a polymer and molecular iodine, and releases molecular iodine. The molecular iodine-infused polymer is prepared by contacting the polymer with a molecular iodine preparation composition.

In certain embodiments, the molecular iodine-infused polymer comprises a first molecular iodine-infused region and a second molecular iodine-infused region. In certain embodiments, molecular iodine from the first molecular iodine-infused region diffuses to the second molecular iodine-infused region while molecular iodine of the second molecular iodine-infused region is released from the polymer. As an example, an embodiment of molecular iodine-infused breast implant showed an oscillatory behavior of I₂ capture/release as its color faded from brown, turned brown, faded, turned brown and faded again (Example 4).

In certain embodiments, the first molecular iodine-infused region has a higher molecular iodine concentration (either mass concentration or volume concentration) than the second molecular iodine-infused region and molecular iodine in the second molecular iodine-infused region is released faster than molecular iodine in the first molecular iodine-infused region. In certain embodiments, the first molecular iodine-infused region has a higher molecular iodine concentration than the second molecular iodine-infused region and molecular iodine in the first molecular iodine-infused region is released faster than molecular iodine in the second molecular iodine-infused region.

In certain embodiments, the polymer is a natural or synthetic polymer. Examples of natural polymers include, without limitation, natural latex and chitosan, which may be further processed or treated. In certain embodiments, examples of the synthetic polymer include, without limitation, silicone, synthetic latex rubber, polyester (e.g., Dacron), and co-polymers thereof.

In certain embodiments, the polymers are medical-grade polymers. In certain embodiments, the molecular iodine-infused polymers are medical-grade polymers.

### II. Molecular Iodine-Infused Articles

Embodiments of the molecular iodine-infused articles are disclosed herein. In certain embodiments, the molecular iodine-infused article comprises one or more molecular iodine-infused polymers. In certain embodiments, the one or more molecular iodine-infused polymers form a coating of the article.

In certain embodiments, the articles are medical-grade articles. In certain embodiments,

the molecular iodine-infused articles are medical-grade articles. In certain embodiments, the molecular iodine-infused article comprises one or more molecular iodine-infused polymers that are medical-grade. In certain embodiments, the molecular iodine-infused article is for medical use. In certain embodiments, the molecular iodine-infused article is a medical device. Examples of the articles of molecular iodine-infused articles include, without limitation, catheters, sutures, grafts, stents, wound dressing material, bandage, artificial skin, implants, packaging to hold sterile medical materials, and polymers that provide a disinfecting atmosphere for materials (e.g., metals, polymers, fabrics, plants, and food) by virtue of being packaged with or placed adjacent to said materials.

Examples of molecular iodine-infused catheters include, without limitation, indwelling catheters, port catheters for, e.g., dialysis.

### III. Molecular Iodine-Infused Products

Embodiments of the molecular iodine-infused products are disclosed. In certain embodiments, the molecular iodine-infused product comprises one or more molecular iodine-infused articles in one or more compartments, and may further include a molecular iodine storage composition. In certain embodiments, the molecular iodine-infused product may comprise additional compartments, the additional compartments may include a molecular iodine pre-treatment composition for pre-treating an article, such as an indwelling urinary catheter before use. When the compartment includes a molecular iodine pre-treatment composition, the product may include an article that may or may not be infused with molecular iodine. The additional compartments may comprise one or more un-infused articles, or a molecular iodine in-use composition for administration to a subject.

In certain embodiments, the molecular iodine in-use composition may be administered to the subject by being applied to the molecular-iodine infused article which is in contact with the subject or will be applied to the subject. For example, the molecular iodine in-use composition may be applied to the outer surface of the molecular iodine-infused article and applied to the subject when the molecular iodine-infused article contacts the subject. Alternatively and additionally, the molecular iodine in-use composition may be administered to the subject by delivering through the molecular iodine-infused article. In certain embodiments, the molecular iodine in-use composition may be administered to the subject after the molecular iodine-infused article is placed in the subject.

The molecular iodine-infused article may be prepared by contacting an un-infused polymeric article with a molecular iodine preparation composition.

In certain embodiments, the molecular iodine storage composition, the pre-treatment composition, the molecular iodine in-use composition, and the molecular iodine preparation composition are embodiments of high molecular iodine concentration compositions disclosed herein. In certain embodiments, the molecular iodine storage composition, the pre-treatment composition, the in-use composition, and the preparation composition are the same. In other embodiments, the molecular iodine storage composition, the pre-treatment composition, the in-use composition, and the preparation composition are different. In some instances, the molecular iodine storage composition can be the same as the preparation composition but in other instances, the preparation composition can contain a higher or lower concentration of molecular iodine. In certain embodiments the articles may be treated with an in-use composition that is contacted to the article after it has been used for a time period. As an example, an indwelling urinary catheter may be initially treated with a preparation composition that contains a very high concentration of molecular iodine and placed in a storage composition that contains a higher or lower concentration of molecular iodine and then an in-use composition with a lower concentration of molecular iodine may be transferred into the interior of said catheter several days after the catheter has been placed in a subject, optionally, the catheter may be bathed in the pre-treatment composition after it is removed from the package and before it is placed in the subject.

In certain embodiments, the molecular iodine-infused product is a sanitizing chamber comprising a first compartment made of one or more molecular iodine-infused polymers. Molecular iodine released from the molecular iodine-infused polymers provides a disinfecting atmosphere for materials (e.g., metals, polymers, fabrics, plants, and food) may disinfect an article that is placed in the sanitizing chamber. In certain embodiments, the first compartment further includes one or more molecular iodine-infused polymers or articles therein. In certain embodiments, the first compartment includes one or more molecular iodine-infused polymers or articles therein but the first compartment itself is not made of a molecular iodine-infused polymer. In certain embodiments, the sanitizing chamber further comprises a second compartment separated from the first compartment by and a physical barrier. The barrier can be breakable to allow fluid communication between the first and second compartments.

In certain embodiments, the compartments of the molecular iodine-infused product comprise one or more material that is dormant to molecular iodine infusion, e.g., without limitation, LLDPE, PTFE, and PET.

### IV. Biostatic Persistent, Biocidal Persistent, Prolonged Biocidal Activity, Iodine Flux Rate, Molecular Iodine Concentration of Molecular Iodine-Infused Polymers, and Articles, and Stability.

Molecular iodine is released from the molecular iodine-infused polymer or article.

In certain embodiments, the molecular iodine-infused polymer or article is biostatic persistent. In certain embodiments, the molecular iodine-infused polymer or article is biocidal persistent. In certain embodiments, the molecular iodine-infused polymer or article has prolonged biocidal activity. In certain embodiments, molecular iodine is released from the molecular iodine-infused polymer or article.

The molecular iodine-infused polymer or article has an iodine flux rate. In certain embodiments, the iodine flux rate of an embodiment of the molecular iodine-infused polymer or article may be measured, e.g., by a method shown in Example 3B. In certain embodiments, the molecular iodine-infused polymer or article has an iodine flux rate sufficient to afford biostatic persistent, biocidal persistent, and prolonged biocidal activity.

In certain embodiments, the iodine flux rate of the molecular iodine-infused polymer or article drops about 90%, about 85%, about 80%, about 75%, about 70%, about 65%, about 60%, about 55%, or about 50% after a time period.

Examples of the iodine flux rate include, without limitation, at least about 0.02, at least about 0.03, at least about 0.08, at least about 0.10, at least about 0.17, at least about 0.19, at least about 0.20, at least about 0.27, at least about 0.45, at least about 0.60, at least about 0.64, at least about 0.69, or at least about 1.08 μg molecular iodine per second per cm² of the molecular iodine-infused polymer or article. Examples of the iodine flux rate include, without limitation, at least about 0.0020, at least about 0.0039, at least about 0.0085, at least about 0.017, at least about 0.029, at least about 0.069, at least about 0.073, at least about 0.078, at least about 0.087, at least about 0.090, at least about 0.11, at least about 0.13, at least about 0.15, at least about 0.19, at least about 0.20, at least about 0.28, at least about 0.30, at least about 0.34, at least about 0.39, at least about 0.41, at least about 0.49, at least about 0.52, at least about 0.547, at least about 0.59, at least about 0.63, at least about 0.74, at least about 1.29, at least about 1.7, at least about 1.81, at least about 3.5, at least about 4.9, at least about 5.0, at least about 5.5, at least about 5.7, at least about 5.8, at least about 6.3, at least about 7.1, at least about 7.2, at least about 7.4, at least about 8.98, at least about 9.4, or at least about 10.94 μg molecular iodine per second per gram of the molecular iodine-infused polymer or article, or ranges therebetween. In certain embodiments, the molecular iodine-infused polymer or article has an iodine flux rate of 0.19 μg of I₂ per second per gram of polymer for at least about 5 hours, 24 hours, 10 days, or 10.4 days. See, e.g., Examples 2 & 3B.

The molecular iodine-infused polymer or article has a molecular iodine mass concentration which may be characterized as the mass of molecular iodine per unit mass of the molecular iodine-infused polymer or article. Examples of the molecular iodine mass concentration includes, without limitation, at least about 13.0 mg/g, 5.16 mg/g, 4.54 mg/g. 2.32 mg/g, and ranges therebetween. See, e.g., Example 3A.

The molecular iodine-infused polymer or article has a molecular iodine volume concentration which may be characterized as the mass of molecular iodine per unit volume of the molecular iodine-infused polymer or article.

In certain embodiments, the molecular iodine-infused polymer or article has one or more biocidal effects selected from the group consisting of biostatic persistent, biocidal persistent, and prolonged biocidal activity for a time period. In certain embodiments, the molecular iodine-infused polymer or article has an iodine flux rate sufficient to afford one or more biocidal effects selected from the group consisting of biostatic persistent, biocidal persistent, and prolonged biocidal effects for a time period. In certain embodiments, the molecular iodine-infused polymer or article has an iodine flux rate or a molecular iodine concentration for a time period. Examples of the time period include, without limitation, about 5 hour to about 11 days, about 24 hours to about 2 weeks, about 10 days to about 2 weeks, at least about 1 min, 5 min, 15 min, 30 min, 1 hr, 1 hr, 1.5 hr, 2 hr, 2.5 hr, 3 hr, 3.5 hr, 4 hr, 4.5 hr, 5 hr, 5.5 hr, 6 hr, 6.5 hr, 7 hr, 7.5 hr, 8 hr, 8.5 hr, 9 hr, 9.5 hr, 10 hr, 10.5 hr, 11 hr, 11.5 hr, 12 hr, 12.5 hr, 13 hr, 13.5 hr, 14 hr, 14.5 hr, 15 hr, 15.5 hr, 16 hr, 16.5 hr, 17 hr, 17.5 hr, 18 hr, 18.5 hr, 19 hr, 19.5 hr, 20 hr, 20.5 hr, 21 hr, 21.5 hr, 22 hr, 22.5 hr, 23 hr, 23.5 hr, 24 hr, 24.5 hr, 25 hr, 25.5 hr, 26 hr, 26.5 hr, 27 hr, 27.5 hr, 28 hr, 28.5 hr, 29 hr, 29.5 hr, 30 hr, 30.5 hr, 31 hr, 31.5 hr, 32 hr, 32.5 hr, 33 hr, 33.5 hr, 34 hr, 34.5 hr, 35 hr, 35.5 hr, 36 hr, 36.5 hr, 37 hr, 37.5 hr, 38 hr, 38.5 hr, 39 hr, 39.5 hr, 40 hr, 40.5 hr, 41 hr, 41.5 hr, 42 hr, 42.5 hr, 43 hr, 43.5 hr, 44 hr, 44.5 hr, 45 hr, 45.5 hr, 46 hr, 46.5 hr, 47 hr, 47.5 hr, 48 hr, 48.5 hr, 49 hr, 49.5 hr, 50 hr, 50.5 hr, 51 hr, 51.5 hr, 52 hr, 52.5 hr, 53 hr, 53.5 hr, 54 hr, 54.5 hr, 55 hr, 55.5 hr, 56 hr, 56.5 hr, 57 hr, 57.5 hr, 58 hr, 58.5 hr, 59 hr, 59.5 hr, 60 hr, 60.5 hr, 61 hr, 61.5 hr, 62 hr, 62.5 hr, 63 hr, 63.5 hr, 64 hr, 64.5 hr, 65 hr, 65.5 hr, 66 hr, 66.5 hr, 67 hr, 67.5 hr, 68 hr, 68.5 hr, 69 hr, 69.5 hr, 70 hr, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, 3 months, 4 months, 5 months, 6 months, 7 months, 8 months, 9 months, 10 months, 11 months, and 1 year, or any ranges therebetween.

### V. Other Properties of the Molecular Iodine-Infused Polymer or Article

In certain embodiments, the shelf-lives of the molecular iodine-infused polymers, molecular iodine-infused articles or molecular iodine-infused products are at least about 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, or 5 years, or ranges therebetween. In certain embodiments, the high molecular iodine concentration compositions or pharmaceutical formulations are stored at room temperature.

### VI. Preparation Methods of Molecular Iodine-Infused Polymer, Article or Product

Embodiments of preparation methods of the molecular iodine-infused polymers or articles are disclosed. In certain embodiments, the method for preparing the molecular iodine-infused polymer or article comprises contacting the polymer or article with a molecular iodine preparation composition.

Embodiments of preparation methods of molecular iodine-infused products are disclosed. In certain embodiments, the method for preparing the molecular iodine-infused product comprises placing the one or more molecular iodine-infused articles in the one or more compartments. In certain embodiments, the method further comprises adding a molecular iodine storage composition into the compartment(s) holding the article(s). In certain embodiments, the method for preparing a molecular iodine-infused product comprises placing one or more molecular iodine-infused catheters in one or more compartments and adding a molecular iodine storage composition into the compartment(s) holding the catheters. In certain embodiments, the preparation methods of molecular iodine-infused products further comprise adding a molecular iodine in-use composition or pre-treatment composition into one or more additional compartments.

In certain embodiments, the molecular iodine-infused polymer or article comprises at least a portion of molecular iodine-infused polymer prepared by contacting the polymer with a molecular iodine preparation composition. Examples of the molecular iodine concentration of the molecular iodine preparation composition include, without limitation, at least about 387 ppm, 500 ppm, 587 ppm, 730 ppm, 738 ppm, 998 ppm, 1,000 ppm, 1,019 ppm, 1,485 ppm, 1,500 ppm, 2,025 ppm, 2,858 ppm, 3,000 ppm, 3,196 ppm, 3,305 ppm, 3,376 ppm, 3,688 ppm, 4,161 ppm, 4,905 ppm, 5,000 ppm, 5,831 ppm, 6,000 ppm, 7,007 ppm, 7,961 ppm, 9,000 ppm, 10,000 ppm, 10,100 ppm, 10,400 ppm, 12,560 ppm, 14,000 ppm, 14,670 ppm, 15,500 ppm, 16,000 ppm, 17,000 ppm, 17,800 ppm, 19,400 ppm, 20,000 ppm, 24,000 ppm, 40,000 ppm, 54,000 ppm, 72,000 ppm, 80,000 ppm, 92,000 ppm, or 100,000 ppm, or ranges therebetween.

Examples of the contact time of the polymer or article with the molecular iodine preparation composition include, without limitation, at least about 30 min., 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 2 days, 3 days, 4 days, 5 days, 6 days, 1 week, eight days, nine days, 10 days, 11 days, 12 days, 2 weeks, 3 weeks, or 4 weeks, or ranges therebetween.

Unless otherwise specified, the molecular iodine storage composition, the molecular iodine pre-treatment composition, the molecular iodine preparation composition, and the molecular iodine in-use compositions are embodiments of the high molecular iodine concentration compositions or pharmaceutical formulations disclosed herein. For an embodiment of molecular iodine-infused product, the molecular iodine storage composition(s), the molecular iodine pre-treatment composition(s), the molecular iodine in-use composition (s) and the molecular iodine preparation composition(s) may be the same or different, may comprise or have the same or different organic carrier(s), and may have the same or different molecular iodine concentration(s).

The one or more molecular iodine-infused polymers of the molecular iodine-infused article may be prepared using the same of different molecular iodine preparation compositions, e.g., the same or different high molecular iodine concentration compositions disclosed herein. For example, without limitation, the one or more molecular iodine preparation compositions may have the same or different molecular iodine concentrations or the same or different organic carriers.

### VII. The High Molecular Iodine Concentration Compositions

In certain embodiments, the high molecular iodine concentration composition comprises:
molecular iodine having concentration of about 387 ppm to about 1,274 ppm, 1,121 ppm to about 1,399 ppm wt/v, about 1,150 ppm to about 1,350 ppm wt/v, about 1,200 ppm to about 1,300 ppm wt/v, or about 1,250 ppm to about 1,275 ppm wt/v, about 1,400 ppm to about 170,000 ppm, about 1,400 ppm to about 160,000 ppm, about 1,400 ppm to about 150,000 ppm, about 1,400 ppm to about 100,000 ppm, about 1,400 ppm to about 68,000 ppm, about 1,400 ppm to about 66,000 ppm, about 1,400 ppm to about 60,000 ppm, about 1,400 ppm to about 55,000 ppm, about 1,400 ppm to about 50,000 ppm, about 1,400 ppm to about 45,000 ppm, about 1,400 ppm to about 40,000 ppm, about 1,400 ppm to about 35,000 ppm, about 1,400 ppm to about 30,000 ppm, about 1,400 ppm to about 25,000 ppm, about 1,400 ppm to about 20,000 ppm, about 1,400 ppm to about 15,200 ppm, about 1,400 ppm to about 15,000 ppm, about 1,400 ppm to about 10,000 ppm, about 1,400 ppm to about 8,703 ppm, about 1,400 ppm to about 8,000 ppm, about 1,400 ppm to about 5,000 ppm, about 1,400 ppm to about 4,077 ppm, about 1,400 ppm to about 2,000 ppm, about 1,400 ppm to about 1,700 ppm, about 1,274 ppm to about 170,000 ppm, about 1,274 ppm to about 160,000 ppm, about 1,274 ppm to about 150,000 ppm, about 1,274 ppm to about 100,000 ppm, about 1,274 ppm to about 68,000 ppm, about 1,274 ppm to about 66,000 ppm, about 1,274 ppm to about 60,000 ppm, about 1,274 ppm to about 55,000 ppm, about 1,274 ppm to about 50,000 ppm, about 1,274 ppm to about 45,000 ppm, about 1,274 ppm to about 40,000 ppm, about 1,274 ppm to about 35,000 ppm, about 1,274 ppm to about 30,000 ppm, about 1,274 ppm to about 25,000 ppm, about 1,274 ppm to about 20,000 ppm, about 1,274 ppm to about 15,200 ppm, about 1,274 ppm to about 15,000 ppm, about 1,274 ppm to about 10,000 ppm, about 1,274 ppm to about 8,703 ppm, about 1,274 ppm to about 8,000 ppm, about 1,274 ppm to about 5,000 ppm, about 1,400 ppm to about 4,077 ppm, about 1,274 ppm to about 2,000 ppm, about 1,274 ppm to about 1,700 ppm, about 1,500 ppm, about 2,000 ppm, about 4,077 ppm, about 4,100 ppm, about 8,200 ppm, about 8,703 ppm, about 15,200 ppm, about 16,500 ppm, or about 33,000 ppm by wt/v; and
an organic carrier having a concentration of no less than about 93.5% wt, no less than about 95% wt, no less than about 98% wt, or no less than about 99% wt of the total weight of the high molecular iodine concentration composition.

In certain embodiments, the organic carrier of the high molecular iodine concentration composition is a pharmaceutically acceptable organic carrier, and the high molecular iodine concentration composition is a pharmaceutical formulation. In certain embodiments, the high molecular iodine concentration pharmaceutical formulation further comprises a second pharmaceutically acceptable carrier.

In certain embodiments, the high molecular iodine concentration composition or pharmaceutical formulation is a solution, a viscous solution, a cream, a lotion, a gel, an ointment, a spray, or a suspension.

Examples of the organic carrier of certain embodiments of the high molecular iodine concentration compositions or pharmaceutical formulations include glycols with molecular weight of less than 300 (e.g., propylene glycol, di-propylene glycol, glycerin), propylene glycol monomethyl ether acetate, dimethyl sulfoxide, alcohols (e.g., ethanol, propanols such as isopropanol and 1-propanol), and any mixtures of the foregoing.

In certain embodiments, the organic carrier of the high molecular iodine concentration compositions or pharmaceutical formulations has a boiling point higher than 100° C. and has a vapor pressure that is less than about 30% of the vapor pressure of molecular iodine. In certain embodiments, the organic carrier comprises one or more anhydrous organic solvents. In certain embodiments, the organic carrier is anhydrous.

In certain embodiments of the high molecular iodine concentration compositions or pharmaceutical formulations, at least about 20%, at least about 35%, at least about 55%, at least about 90% of all iodine species is molecular iodine.

In certain embodiments, the high molecular iodine concentration compositions or pharmaceutical formulations is non-aqueous. In certain embodiments, the high molecular iodine concentration compositions or pharmaceutical formulations is substantially non-aqueous. In certain embodiments, the high molecular iodine concentration compositions or pharmaceutical formulations is water-free. In certain embodiments, the high molecular iodine concentration compositions or pharmaceutical formulations is substantially water-free.

In certain embodiments, the high molecular iodine concentration compositions or pharmaceutical formulations has a water content of no more than about 5% wt, no more than about 4.5% wt, no more than about 4% wt, no more than about 3.5% wt, no more than about 3% wt, no more than about 2.5% wt, no more than about 2% wt, no more than about 1.9% wt, no more than about 1.8% wt, no more than about 1.7% wt, no more than about 1.6% wt, no more than about 1.5% wt, no more than about 1.4% wt, no more than about 1.3% wt, no more than about 1.2% wt, no more than about 1.1% wt, no more than about 1% wt, no more than about 0.9% wt, no more than about 0.8% wt, no more than about 0.7% wt, no more than about 0.6% wt, no more than about 0.5% wt, no more than about 0.4% wt, no more than about 0.3% wt, no more than about 0.2% wt, or no more than about 0.1% wt of the total weight of the high molecular iodine concentration compositions or pharmaceutical formulations.

In certain embodiments, the high molecular iodine concentration compositions or pharmaceutical formulations are alcohol-free. In certain embodiments, the high molecular iodine concentration compositions or pharmaceutical formulations are substantially alcohol-free.

In certain embodiments, the high molecular iodine concentration compositions or pharmaceutical formulations has an alcohol content of no more than about 50% wt, no more than about 40% wt, no more than about 30% wt, no more than about 20% wt, no more than 10% wt, no more than about 5% wt, no more than about 4.5% wt, no more than about 4% wt, no more than about 3.5% wt, no more than about 3% wt, no more than about 2.5% wt, no more than about 2% wt, no more than about 1.9% wt, no more than about 1.8% wt, no more than about 1.7% wt, no more than about 1.6% wt, no more than about 1.5% wt, no more than about 1.4% wt, no more than about 1.3% wt, no more than about 1.2% wt, no more than about 1.1% wt, no more than about 1% wt, no more than about 0.9% wt, no more than about 0.8% wt, no more than about 0.7% wt, no more than about 0.6% wt, no more than about 0.5% wt, no more than about 0.4% wt, no more than about 0.3% wt, no more than about 0.2% wt, or no more than about 0.1% wt of the total weight of the high molecular iodine concentration compositions or pharmaceutical formulations.

In certain embodiments, the high molecular iodine concentration composition or pharmaceutical formulation is complexed iodine -free. In certain embodiments, the high molecular iodine concentration composition or pharmaceutical formulation is substantially complexed iodine-free.

In certain embodiments, the high molecular iodine concentration compositions or pharmaceutical formulation has a total concentration of complexed iodine is no more than about 10% of all iodine species, no more than about 25% of all iodine species, no more than about 50% of all iodine species, no more than about 75% of all iodine species, or no more than about 90% of all iodine species.

In certain embodiments, the high molecular iodine concentration compositions or pharmaceutical formulations further comprise one or more additives. Examples of the one or more additives are, without limitation, iodide, quaternary amines, cationic polymers, anionic polymers, gelling agents, additive polymers, viscosity enhancing agents, unsaturated fatty acids, desiccants, and fragrances.

In certain embodiments, the viscosities of the high molecular iodine concentration compositions or pharmaceutical formulations are no greater than about 100,000 Centipoise (cps), no greater than about 50,000 cps, no greater than about 1,000 cps, or no greater than about 500 cps. In certain embodiments, the viscosities of the high molecular iodine centration compositions or pharmaceutical formulations is about 2,000 cps.

Examples of additive polymers for use in the high molecular iodine concentration compositions or pharmaceutical formulations include carbopols and HPMA polymers.

An example of desiccants for use in the high molecular iodine concentration compositions or pharmaceutical formulations includes zeolites.

In certain embodiments, the high molecular iodine concentration compositions or pharmaceutical formulations further comprise an unsaturated fatty acid that imparts a long-lasting residual bactericidal activity. Examples of the unsaturated fatty acids include lactic acid, myristic acid, 1-monolaurin, dodeconic acid and caprylic acid. Lauric acid, latic acid and caprylic acid can be incorporated directly into propylene glycol.

### VIII. Uses of the Molecular Iodine-Infused Polymers, Molecular Iodine-Infused Articles, or Molecular Iodine-Infused Products.

Embodiments of the molecular iodine-infused polymers, molecular iodine-infused articles, and molecular iodine-infused products have not only the known uses for molecular iodine, but also unexpected uses due to the extended and sustained release and delivery of molecular iodine.

### A. Killing or Inhibiting the Growth of Microorganism

Another aspect of the invention provides a method of treating or preventing a condition associated with a microorganism in a subject comprising contacting the subject with one or more of the molecular iodine-infused polymers or molecular iodine-infused articles or molecular iodine-infused products.

In certain embodiments, the one or more of the molecular iodine-infused polymers or molecular iodine-infused articles remain in the subject for a time period that the one or more of the molecular iodine-infused polymers or molecular iodine-infused articles have one or more biocidal effects selected from the group consisting of biostatic persistent, biocidal persistent, and prolonged biocidal activities. In certain embodiments, the one or more of the molecular iodine-infused polymers or molecular iodine-infused articles remain in the subject for a time period that the one or more of the molecular iodine-infused polymers or molecular iodine-infused articles have an iodine flux that is sufficient to afford biostatic persistent, biocidal persistent, and prolonged biocidal activity. In certain embodiments, the one or more of the molecular iodine-infused polymers or molecular iodine-infused articles remain in the subject for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, or 3 months.

In certain embodiments, the microorganism is killed at the contact site or in vicinity of the contact site. In certain embodiments, the growth rate of the microorganism is reduced at the contact site or in vicinity of the contact site.

In certain embodiments, the method further comprises delivering a therapeutically effective amount or prophylactically effective amount of molecular iodine, high molecular iodine concentration composition, or high molecular iodine concentration pharmaceutical formulation via the molecular iodine infused polymers or articles. In certain embodiments, the method further comprising delivering a molecular iodine in-use composition to the subject via the one or more of the molecular iodine-infused polymers or molecular iodine-infused articles. For example, the molecular iodine in-use composition may be delivered through the molecular iodine-infused catheter. In certain embodiments, the delivery happens after the one or more of the molecular iodine-infused polymers or molecular iodine-infused articles are placed in the subject. In certain embodiments, the delivery happens multiple times at the same interval or at different intervals between each delivery. In certain embodiments, the delivery happens at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, or 3 months after the one or more of the molecular iodine-infused polymers or molecular iodine-infused articles are placed in the subject.

In certain embodiments, the microorganism is present at the contact site or in vicinity of the contact site in or on the subject. In certain embodiments, a biofilm is present at the contact site or vicinity of the contact site. In certain embodiments, more than one species of microorganism are present at the contact site or vicinity of the contact site. In certain embodiments, the biofilm comprises more than one species of microorganism, e.g., 2, 3, 4, 5, or 6 species of microorganism (See Example 2(A)).

Examples of the conditions treatable or preventable include, without limitation, infections caused by microorganism, e.g., urinary tract infection, breast implant infections, wound infections. In certain embodiments, the infection is a chronic infection. In certain embodiments, the infection is resistant infection. In certain embodiments, the infection is a chronic urinary tract infection.

In certain embodiments, the subject is a human.

In certain embodiments, the condition to be treated or prevented is a tissue condition associated with the microorganism. In certain embodiments, the tissue is a mucosal tissue or a cutaneous tissue.

In certain embodiments, the mucosal tissue surrounds or is in a biological cavity. Examples of biological cavities include eye cavity, ear cavity, oral cavity, nasal cavity, vaginal cavity, rectal cavity, and urethral cavity.

In certain embodiments, the microorganisms to be killed of growth of which to be inhibited include one or more species selected from the group consisting of Candida albicans, C. freundii, Escherichia coli, E. faecalis, Enterococcus faecium, Klebsiella pnuemoniae, Proteus mirabilis, Pseudomonas aeruginosa, Staphylococcus aureus, or Staphylococcus epidermidis.

In certain embodiments, the microorganisms to be killed of growth of which to be inhibited include one or more species selected from the group consisting of S. aureus, P. aeruginosa, E. coli, E. faecalis, and C. freundii.

In certain embodiments, the microorganisms to be killed of growth of which to be inhibited include Staphylococcus aureus.

Examples of the microorganism to be killed or growth of which to be inhibited include virus, bacteria, fungus, and protozoa.

Examples of bacteria to be killed or growth of which to be inhibited include gram-positive and gram-negative bacteria, e.g., Bacillus oleronius, C. freundii, E. faecalis, Enterococcus faecium, Klebsiella pnuemoniae. Proteus mirabilis, Pseudomonas aeruginosa, Staphylococcus aureus, Streptococcus pyogenes, Erysipelothrix rhusiopathiae, Mycobacterium tuberculosis, Mycobacterium bovis, Escherichia coli, Extended Spectrum Beta Lactamase resistant E. coli (ESBL), Shigella flexneri, Staphylococcus aureus, Staphylococcus epidermidis, Serratia marcescens, Vibrio cholera, MRSA, Salmonella enterica, Gonorrhea, Syphilis, Shewanella algae, Shewanella putrefaciens, Chlamydia, Chlamydia trachomatis, Chlamydia pneumoniae, Chlamydia psittacci, Aeromonas hydrophila, Vibrio species, Pasteurella multocida, Stapylococcus species, Corynebacterium species, Pripionibacterium species, and antibiotic resistant bacteria, e.g., antibiotic resistant flesh eating bacteria.

Examples of conditions associated with bacteria to be treated or prevented include tuberculosis, periodontitis, acne (e.g., Propionibacterium acnes), rosacea, impetigo, cellulitis, folliculitis, blepharitis (e.g., anterior blepharitis, posterior blepharitis, rosacea blepharitis), bacterial conjunctivitis, blepharoconjunctivits, bacterial corneal ulceration, post-operative endophthalmitis, endophthalmitis after intravitreal or intracameral injection, and infections caused by the bacterium (e.g., urinary tract infections).

Examples of fungus to be killed or growth of which to be inhibited include Apophysomyces variabilis, Aspergillus, Basidiobolus ranarum, Blastomyces dermatitidi, Coccidioides (e.g., Coccidioides posadasii, Coccidioides immitis), Conidiobolus (e.g., Conidiobolus coronatus, Conidiobolus incongruous), Epidermophyton, Fonsecaea (e.g., Fonsecaea pedrosoi, Fonsecaea compacta), Fusarium, Geotrichum candidum, Herpotrichiellaceae (e.g., Exophiala jeanselmei), Histoplasma (e.g., Histoplasma capsulatum, Histoplasma duboisii), Hortaea werneckii, Lacazia (e.g., Lacazia loboi), Hyalohyphomycosis, Lichtheimia corymbifera, Malassezia furfur, Microsporum (e.g., Microsporum canis, Microsporum gypseum), Mucor indicus, onychomycosis (e.g., Distal subungual onychomycosis, Proximal subungual onychomycosis), Phialophora verrucose, Piedraia hortae, Pityrosporum, Pseudallescheria boydii, Rhizopus oryzae, Sporothrix schenckii, Syncephalastrum racemosum, Talaromyces marneffei, Trichophyton (e.g., Trichophyton mbmm, Trichophyton mentagrophytes), and yeast (e.g., Candida such as Candida albicans, Candida glabrata, Candida tropicalis, Candida lusitaniae; Cryptococcus neoformans; Pneumocystis such as Pneumocystis jirovecii).

Examples of conditions associated with fungus to be treated or prevented are Alternariosis, black Piedra, blastomycosis, chromoblastomycosis, conidiobolomycosis, favus, fungal folliculitis, fungal corneal ulceration, Lobomycosis, onychomycosis, Otomycosis, Phaeohyphomycosis Pityrosporum folliculitis, ringworm, tinea (e.g., tinea pedis, tinea cruris, tinea barbae, tinea manuum, tinea unguium, tinea unguium, tinea faciei, tinea versicolon, tinea nigra, tinea corporis gladiatorum, tinea imbricate, tinea incognito), yeast infection (e.g., seborrheic dermatitis, vaginal yeast infections).

Examples of protozoa to be killed or growth of which to be inhibited include Acanthamoeba, Leishmania parasites, trypanosoma, Entamoeba histolytica, and Toxoplasma gondii.

Examples of conditions associated with protozoa to be treated or prevented include Acanthamoeba infections (e.g., Acanthamoeba corneal ulceration), Acanthamoeba keratitis, Leishmaniasis, trypanosomiases, Amebiasis, and Toxoplasmosis.

### C. Uses on Wound-Healing or Scar-Prevention

Another aspect of the invention relates to a method of fostering wound-healing or preventing a scar of a subject comprising contacting the wound with one or more of the molecular iodine-infused polymers or molecular iodine-infused articles (e.g., wound dressing, sutures, artificial skin) or molecular iodine-infused products.

In certain embodiments, the molecular iodine-infused polymer or article is applied to a wound or tissue in proximity of the wound. In certain embodiments, the tissue is a mucosal tissue or a cutaneous tissue.

In certain embodiments, the wound to be healed is healed with a scar less severe than a similar wound healed without the treatment. In certain embodiments, the scar is less severe as characterized by one or more improvements, e.g., without limitation, reduction of the scar height, reduction of the scar surface, reduction of the thickness of the scar, improvement of the pliability of the scar, improvement of the texture of the scar, reduction of pigmentation of the scar, and reduction of vascularity of the scar. See, e.g., Fearmonti et al., “A Review of Scar Scales and Scar Measuring Devices,” Eplasty, 2010:10 e43, which is incorporated by reference (https://www.ncbi.nlm.nih.gov/pmc/articles/PMC2890387/, attached as Appendix I). In certain embodiments, the one or more improvements of the scar are at least about 10%, about 10% to about 100%, at least about 20%, at least about 30%, at least about 40%, at least about 50%, at least about 60%, at least about 70%, at least about 80%, at least about 90%, at least about 95%, or at least about 99%. In certain embodiments, the reduction of the scar height, the reduction of the scar surface, or the reduction of the thickness of the scar are/is at least about 10%, about 10% to about 100%, at least about 20%, at least about 30%, at least about 40%, at least about 50%, at least about 60%, at least about 70%, at least about 80%, at least about 90%, at least about 95%, or at least about 99%.

In certain embodiments, the wound to be healed in presence of the molecular iodine-infused polymer or article is healed without a visible scar.

In certain embodiments, the wound to be healed in presence of the molecular iodine-infused polymer or article is healed in a shorter time period in the presence of the molecular iodine-infused polymer or article compared to similar wound healed without the presence of the molecular iodine-infused polymer or article.

In certain embodiments, the wound to be healed in presence of the molecular iodine-infused polymer or article is inflicted by a cut, a friction, cold, heat, radiation (e.g., sunburn), a chemical, electricity, a microorganism infection, pressure, or a condition of the subject (e.g., diabetes). For example, in certain embodiments, the subject is diabetic (i.e., a subject having a condition, and the condition is diabetes). Examples of wounds in a diabetic subject include bullosis diabeticorum (diabetic blisters), eruptive xanthomatosis, and neuropathic ulcers (e.g., diabetic foot ulcers). In certain embodiments, the wound is a decubitus ulcer (i.e., pressure ulcer, pressure sore, or bedsore). In certain embodiments, the wound is a burn wound.

### D. Uses for Pre-, Post- and Intra-Operative Infection Prevention

Provided are embodiments of a method of treating a surgical site to foster healing, or prevent infection in a subject in need comprising contacting the subject with one or more of the molecular iodine-infused polymers or molecular iodine-infused articles (e.g., wound dressing, sutures, and artificial skin) or molecular iodine-infused products. In certain embodiments, the method of treating a surgical site is performed as a pre-surgical treatment, a post-surgical treatment, or a treatment during a surgery.

A surgical site refers to an incision site on a subject, or any part of a subject's anatomy in organs or spaces which were opened or manipulated during a surgery. In certain embodiments, the incision site includes a superficial incision site, e.g., involving skin or subcutaneous tissue. In certain embodiments, the incision site includes a deep incision site, e.g., in tissues deeper than the skin or subcutaneous tissue, such as fascial and muscle layers.

In certain embodiments, the molecular iodine-infused polymer or article is applied to a surgical site or tissue in proximity of the surgical site.

In certain embodiments, the molecular iodine-infused polymer or article is applied to a surgical site pre-surgical, post-surgical, or during surgery. Examples of surgery include pleurodesis procedure, cytoreductive surgery, thoracic surgery, esophageal resection, complete resection or pleural reductive surgery for thymoma, primary functional endoscopic sinus surgery, spinal surgery, and colonic resection.

In certain embodiments, the molecular iodine-infused polymer or article is administered as an eyelid antisepsis prior, during, or after an eye surgery (e.g., cataract surgery) or other procedures on or proximate to an eye (e.g., intravitreal injection, intracameral injection). In certain embodiments, the eyelid antisepsis is applied to eyelid by hand or by the molecular iodine-infused polymer or molecular iodine-infused article (e.g., an eyelid wipe).

In certain embodiments, the molecular iodine-infused polymer or article is applied to a surgical site comprising a chest cavity (i.e., a space between a subject's lung and chest wall). For example, the molecular iodine-infused polymer or molecular iodine-infused article may be administered before, during, or after a pleurodesis procedure in a subject in need.

### E. Uses for Sanitizing One or More Articles

Provided herein are embodiments of sanitizing one or more articles or materials (e.g., metals, polymers, fabrics, plants, and food) comprising placing the one or more articles or materials adjacent to one or more molecular iodine-infused polymers or one or more molecular iodine-infused articles. In certain embodiments, the one or more articles or materials are placed in a sealed compartment comprising the one or more molecular iodine-infused polymers or one or more molecular iodine-infused articles. In certain embodiments, the sealed compartment is made of the one or more molecular iodine-infused polymers. In certain embodiments, the sealed compartment comprises one or more molecular iodine-infused polymers or articles placed therein.

IX. Preparation of High Molecular Iodine Concentration Compositions Provided are also methods for preparing the high molecular iodine concentration compositions. In certain embodiments, the high molecular iodine concentration composition is prepared by dispersing iodine into the organic carrier. In certain embodiments, the preparation method comprises:
1) dispersing an iodine composition in a first organic solvent to prepare a first iodine dispersion; and
2) dispersing the first iodine dispersion into a second organic solvent to provide the high molecular iodine concentration composition as desired.

In certain embodiments, all organic solvents are anhydrous.

In certain embodiments, the first and the second organic solvents are the same.

In certain embodiments, the first and the second organic solvents are different, and the first organic solvent has better solubility of iodine than the second organic solvent.

In certain embodiments, the first organic solvent has a viscosity less than 10 centipoise.

In certain embodiments, the first iodine dispersion has a molecular iodine concentration at least ten times of that of the high molecular iodine concentration composition.

Examples of the first organic solvent include, without limitation, propylene glycol, alcohols (e.g., ethanol and propanols such as isopropanol and 1-propanol), and dimethyl sulfoxide. Examples of the second organic solvent include, without limitation, glycols with molecular weight of less than 300 (e.g., glycerin, propylene glycol), and combinations thereof.

In certain embodiments, the preparation method comprises:
1) mixing elemental iodine in ethanol to prepare a first iodine ethanol concentrate that has I₂ concentration of about 5% to about 40% wt; and
2) mixing the first iodine ethanol concentrate into a glycol with molecular weight of less than 300 (e.g., glycerin, propylene glycol, and combinations thereof) to provide the high molecular iodine concentration composition as desired.

In certain embodiments, the preparation of the high molecular iodine concentration composition comprises mixing the first iodine ethanol concentrate with the glycol with molecular weight of less than 300 for at least twenty minutes.

The above disclosure and the following examples are illustrative of the teachings of this application and are not meant to limit the scope and application of the invention. The examples and embodiments are for illustrative and will suggest modifications to persons skilled in the art which are included within the spirit and purview of this application and scope of the appended claims.

### EXAMPLES

### Example 1

### Overview of Examples Regarding Preparation and Characterizations of Embodiments of Molecular Iodine-Infused Polymers and Articles

Unless otherwise specified, embodiments of molecular iodine-infused polymers and articles referred to in the EXAMPLES section were prepared by contacting the polymers and articles with a specified molecular iodine preparation composition. The molecular iodine preparation compositions may be high molecular iodine concentration compositions described herein. The molecular iodine preparation compositions may have various organic carriers (e.g., propylene glycol, glycerin, ethanol, dimethyl sulfoxide, and isopropanol) and various molecular iodine concentrations (e.g., 25 ppm, 99 ppm, 100 ppm, 136 ppm, 251 ppm, 301 ppm, 373 ppm, 387 ppm, 500 ppm, 587 ppm, 730 ppm, 738 ppm, 998 ppm, 1,000 ppm, 1,019 ppm, 1,485 ppm, 1,500 ppm, 2,025 ppm, 2,858 ppm, 3,000 ppm, 3,196 ppm, 3,305 ppm, 3,376 ppm, 3,688 ppm, 4,161 ppm, 4,905 ppm, 5,000 ppm, 5,831 ppm, 6,000 ppm, 7,007 ppm, 7,961 ppm, 9,000 ppm, 10,000 ppm, 10,100 ppm, 10,400 ppm, 12,560 ppm, 14,000 ppm, 14,670 ppm, 15,500 ppm, 16,000 ppm, 17,000 ppm, 17,800 ppm, 19,400 ppm, 20,000 ppm, 24,000 ppm, 40,000 ppm, 54,000 ppm, 72,000 ppm, 80,000 ppm, 92,000 ppm, or 100,000 ppm) for various periods of time (e.g., overnight, 30 minutes, 16 hours to 24 hours, 1 day, 2 days, 3 days, 4 days, 5 days, 6 days, 7 days, 8 days, 9 days, 10 days, 11 days) to saturation, rinsed with water, and patted dry.

Some of the treated polymers or articles turned from clear to dark brown during the treatment with the molecular iodine preparation compositions. Molecular iodine was released from the embodiments of molecular iodine-infused polymers or articles as visualized by the fading of their colors to clear or a color that no longer change after various period of time (e.g., 250.75 hours, 208 hours, 83.75 hours, 69.5 hours, about 65 hours, 37.5 hours, about 19 hours, or about 12 hours). See, e.g., Example 4.

The iodine flux rates and the molecular iodine concentration of the embodiments of the molecular iodine-infused polymers and articles were measured as set forth in Example 3.

Biocidal effects (e.g., biostatic persistence, biocidal persistence, and prolonged biocidal activity) of embodiments of molecular iodine-infused polymers and articles were observed when the molecular iodine-infused polymers and articles were exposed to various microorganism challenges. Examples of microorganism challenges include, without limitation, Candida albicans, C. freundii, Escherichia coli, E. faecalis, Enterococcus faecium, Klebsiella pnuemoniae, Proteus Pseudomonas aeruginosa, Staphylococcus aureus, or Staphylococcus epidermidis streaked on agar plates such as MacConkey agar plates, or presented in buffer or in biofilms (Example 2). Iodine flux rates sufficient to provide biocidal activity, biostatic persistence, biocidal persistence, or prolonged biocidal activity were measured.

Furthermore, polymers or articles treated with high molecular iodine concentration compositions disclosed herein provide iodine flux that provided a long term I₂ flux as compared to the limited time exhibited by polymers or articles treated with aqueous solutions of molecular iodine. See, Tables 3(B)(ii)-(iii).

### Example 2

### Biocidal Effects of the Molecular Iodine-Infused Polymers And Articles (e.g., Cathters)

Biocidal effects of an embodiment of the molecular iodine-infused polymer or article were evaluated by subjecting a sample of the embodiment of the molecular iodine-infused polymer or article to a microorganism challenge for 24 hours.

### 2(A). Quantitative Biocidal Effects of Embodiments of the Molecular Iodine-Infused Silicone Catheter

See Table 2(A) for summary of biocidal effects of various embodiments of the molecular iodine-infused catheter to microorganism challenges.

Preparation of embodiments of molecular iodine-infused polymers and molecular iodine-infused catheters: Polymers or catheters suitable were treated with various molecular iodine preparation compositions as specified in Table 2(A) at room temperature until the polymers or catheters were saturated with molecular iodine (e.g., 5 days) to provide molecular iodine-infused polymers or molecular iodine-infused catheters.

### 2(A)(i) Quantitative Biocidal Effects to Biofilms:

A wound biofilm model (Nedelea A G, Plant R L, Robins L I, Maddocks S E. Testing the efficacy of topical antimicrobial treatments using a single, two-, and five-species chronic wound biofilm model. J. Appl. Microbiol. 2022 January; 132(1):715-724. doi: 10.1111/jam.15239. Epub 2021 Aug. 10. PMID: 34319637) was used to evaluate the biocidal effects of various embodiments of molecular iodine-infused gels, iodine-infused silicone catheter material, and I₂-glycerin solution (up to 4,905 ppm I₂). Biofilms were grown for at least 48 hours and a time of 24 hours was used to evaluate the biocidal activity of the iodine-based materials. P. aeruginosa were used to prepare single species biofilms (Table 2(A)(i)-1). The 2-species biofilm (Table 2(A)(i)-2) was prepared with both P. aeruginosa and S. aureus. The 2-species biofilm was tested against molecular iodine-infused catheter material of silicone (Shenzhen Laimeisi Silicone Industry Co., Ltd., Shenzhen, China, hereinafter RSH, 1.016 mm thick) covered with a piece of laboratory-grade PTFE film (Oil Slick Pad, Bellingham, Wash., USA, 8 mm diameter). The PTFE served to limit loss of molecular iodine to the atmosphere over the 24-hour exposure time (Table 2(A)(i)-2). The same catheter material without molecular iodine treatment (untreated catheters and untreated RSH) was used as a negative control with the 2-species biofilm.

A 5-species biofilm was prepared as described by Nedelea A G et al. The 5-species biofilm is known to be very resistant to antimicrobials. The log reduction (log 10, unless otherwise specified) observed against a single species biofilm with the I₂-glycerin material was modest but demonstrated that biofilm eradication was proportional to the concentration of I₂ exposure. silicone A 10,000 ppm I₂ gel was prepared and tested to be much more effective than the I₂ glycerin materials tested, as it eliminated almost all the biofilm, i.e. (>99.999%)(see Table 2(A)(i)-3). Surprisingly, the iodine-infused silicone catheter was even more effective than the gel, as there was complete eradication of the 5-species biofilm. Biofilm eradication with the iodine-infused silicone catheter material in the 5-species biofilm was observed to be greater than 3 logs which is higher than what is observed with other commercial products.

TABLE 2(A)(i)-1
Quantitative biocidal effects of various embodiments of the molecular
iodine-infused silicone catheters on biofilms with P. aeruginosa.

| | I₂ conc. (ppm) | Final | | |
|---|---|---|---|---|
| | in molecular | microorganism | | |
| | iodine preparation | conc. after 24 | Log | Log |
| | composition | hr exposure | reduction | reduction |
| Samples | (glycerin) | (Log cfu/mL) | (cfu/mL) | (cfu/mL) |
| I₂- | 4,905 | 6.928 | 1.639 | NA |
| infused | | | | |
| Catheter^{a} | 1,485 | 7.791 | 0.776 | NA |
| | 99 | 8.116 | 0.45 | NA |
| Control^{b} | 0 | (8.56) ± 0.184 | 0 | NA |

^{a}Bardia Foley 100% silicone catheter, 30 cc, size 24Fr (Ref#806324, Lot#NGFS2195), density of about 1.2 g/cm³.
^{b}Untreated catheter.

TABLE 2(A)(i)-2
Quantitative biocidal effects of various embodiments
of the molecular iodine-infused silicone materials on biofilms
with both P. aeruginosa and S. aureus.

| | | I₂ conc. (ppm) | | |
|---|---|---|---|---|
| | | in molecular | | |
| | | iodine | Microorganism conc. after | |
| | | preparation | 24-hour exposure to the | |
| | Size/shape of | composition | mix biofilm (Log cfu/mL) | |
| Samples | the sample | (Glycerin) | P. aeruginosa | S. aureus |
| I₂- | flat circular | 10,400 | 0 | 0 |
| infused | silicone 8 mm | | | |
| RSH | diameter | | | |
| Untreated | flat circular | N/A | 5.833 | 4.471 |
| RSH | silicone 8 mm | | | |
| | diameter | | | |

TABLE 2(A)(i)-3
Quantitative biocidal effects of various embodiments of the molecular
iodine-infused silicone catheters^{a} on biofilms with five species

| | Species | | | | |
|---|---|---|---|---|---|
| | S. aureus | P. aeruginosa | E. coli | E. faecalis | C. freundii |
| | Final Log | Final Log | Final Log | Final Log | Final Log |
| | (cfu/mL) | (cfu/mL) | cfu/mL | cfu/mL | cfu/mL |
| Control^{b} | 7.938 | 7.929 | 6.866 | 7.633 | 7.069 |
| Control^{c} | 7.928 | 7.901 | 7.256 | 7.598 | 7.294 |
| I₂-gel^{d} | 5.367 (2.57) | 5.58 (2.34) | 4.825 (2.04) | 6.679 (0.954) | 4.368 (2.70) |
| (Log kill) | | | | | |
| I₂- | 4.876 (3.06) | 4.786 (3.14) | 3.470 (3.39) | 4.398 (3.23) | 3.515 (3.55) |
| silicone^{e} | | | | | |
| (Log kill) | | | | | |

^{a}Bardia Foley 100% silicone catheter, 30 cc, size 24Fr (Ref#806324, Lot#NGFS2195), density of about 1.2 g/cm³.
^{b}Blank, biofilm without treatment.
^{c}Negative control, biofilm treated with untreated silicone catheter.
^{d}10,000 ppm I₂.
^{e}17,000 ppm I₂.

### 2(A)(ii) Biocidal Effects Measured Under Dynamic Contact Conditions by ASTM Method E2149-20

Silicone catheters (Bardia Foley 100% Silicone Catheter, 30 cc, Size 24Fr (Lot# NGFS2195) were treated with various molecular iodine preparation compositions as specified in Table 2(A)(ii) to molecular iodine saturation (e.g., 5 days) to provide molecular iodine-infused catheters.

ASTM Method E2149-20 was used to compare the antimicrobial activity of an I₂-infused silicone catheter to the identical catheter that was not treated (negative control). ASTM Method E2149-20 evaluates the antimicrobial activity of a material that is in direct contact with test organisms. The standard organisms used in this test were Escherichia coli NCTC 12241 (ATCC 25922) and Staphylococcus aureus ATCC 6538. Suspensions of the test organisms in a buffer solution were prepared at a concentration that was at least 1.5× 10E5 cfu/mL. Molecular iodine-infused catheters and untreated catheters (negative control) were placed in a suspension of each bacteria and mixed for 24 hours after which an aliquot of the liquid was removed and enumerated on agar plates in triplicate. A “time 0” sample of each bacterial suspension was taken prior to contact with either the molecular iodine-infused catheter material or untreated catheter material to evaluate the starting concentration of each organism. The number of viable organisms was determined, and the reduction or kill of bacteria was calculated by comparison the bacterial concentration of the untreated catheter to the treated catheter.

TABLE 2(A)(ii)
Quantitative biocidal effects of an embodiment of the molecular iodine-
infused silicone catheters^{a} under dynamic contact condition for 24 hours
with E. coli and S. aureus (ASTM Method E2149-20).

| | Average | | |
|---|---|---|---|
| | cfu/mL (n = 3) | Log cfu/mL | Log Kill cfu/mL |
| | Escherichia coli | | |
| Time 0 control | 260,000 | 5.41 | NA |
| Untreated catheter^{a} 24 | 343,333 | 5.54 | NA |
| hr | | | |
| 12 Treated catheter^{a} 24 | 30^{b} | 1.48 | >4.06 |
| hr | | | |
| | Staphylococcus aureus | | |
| Time 0 control | 376,667 | 5.58 | NA |
| Untreated catheter^{a} 24 | 326,667 | 5.51 | NA |
| hr | | | |
| I2 Treated catheter^{a} 24 | 30^{b} | 1.48 | >4.03 |
| hr | | | |

^{a}Bardia Foley 100% Silicone Catheter, 30 cc, 24Fr (Lot#NGFS2195).
^{b}<30 cfu/mL for undiluted samples were assigned a 30 cfu/mL value.

Table 2(A)(ii) demonstrates that no antimicrobial activity was observed from the untreated catheter. In contrast, the molecular iodine-infused catheter demonstrated a potent antimicrobial activity as it killed greater than 4 logs of each of the test organisms.

2(B) Biocidal Effects of Molecular Iodine-Infused Catheters Or Molecular Iodine-infused Materials on E. coli Streaked Agar Plates.

Preparation of E. coli streaked agar plates: E. coli in phosphate buffer (0.1 M, pH 5.0, with optical densities between 0.85 and 0.95 at 600 nm. were diluted 1/1,000, and then streaked (10 μL) on a MacConkey agar plate.

Preparation of molecular iodine-infused catheters and materials: Catheters or catheter materials were treated with various molecular iodine preparation compositions as specified in Tables 2(B)-1 to 2(B)-2, and allowed to reach saturation with respect to the I₂ concentration (e.g., 4 days) to provide molecular iodine-infused catheters or molecular iodine-infused catheter materials.

Biocidal effects of molecular iodine-infused catheters or molecular iodine-infused materials on the E. coli streaked agar plates: A sample of molecular iodine-infused catheters or molecular iodine-infused catheter materials with a size and shape specified in Tables 2(B)-1 to 2(B)-2, was removed from the solution, washed with distilled water, dried on a paper towel and then placed on an E. coli streaked agar plate to evaluate the biocidal effects of the iodine-infused polymer. Untreated catheter or catheter materials were used as negative controls; E. coli streaked agar plates with no treatment (including untreated catheter) were used as a blank control. The E. coli streaked agar plates with the sample catheters, negative control and blank control were incubated at 37° C. for 24 hours and imaged to assess biocidal effects of the tested molecular iodine-infused catheters or molecular iodine-infused materials. No colony (bacterial growth) was observed in contact with the molecular iodine-infused catheters or molecular iodine-infused materials, although a dose-dependent pattern of bacteria reduction in the vicinity of the molecular iodine-infused catheters or molecular iodine-infused materials was observed (Tables 2(B)-1 to 2(B)-3). The observations reported in Table 2(B)-1 demonstrate that an iodine flux of or above 0.020 ug I_{2/}gram/second could kill bacteria.

Measurement of I₂ Flux (μg I_{2/}gram polymer/sec) shown in Tables 2(B)-1 and 2(B)-3 was performed as described in Example 3(B)(i). The units of I₂ flux for tubular sections of catheter material (Tables 2(B)-1 to 2(B)-3) were calculated in μg I₂ per gram of polymer per second.

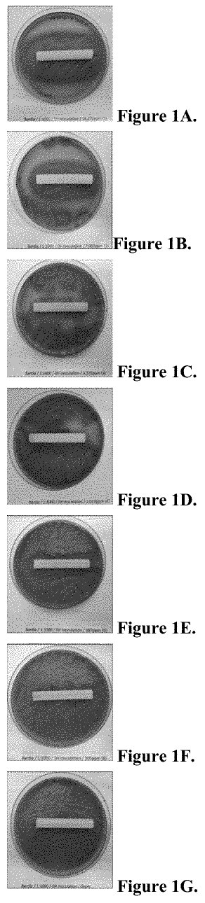

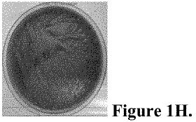

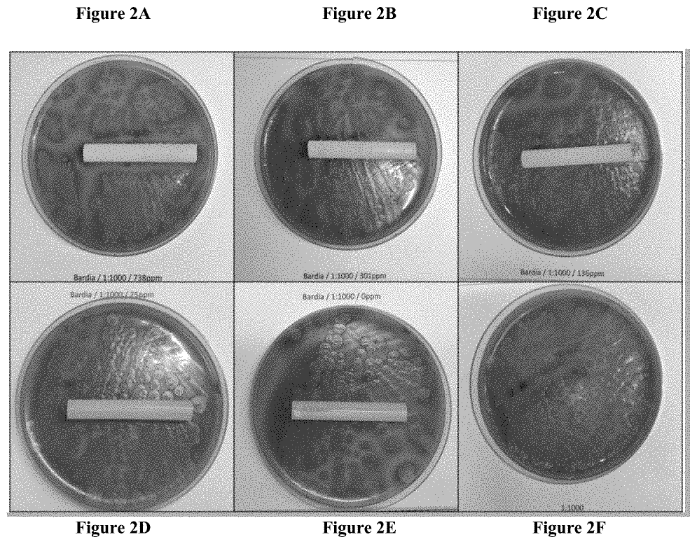

TABLE 2(B)-1
Biocidal effects of various embodiments of the molecular iodine-
infused silicone catheters (Bardia^{a} catheter tube material, 5
cm in length) on E. coli streaked agar plates (E. coli
solution OD600 = 0.926 before 1/1000 dilution).

| Molecular iodine preparation | | |
|---|---|---|
| composition, I₂ conc. | | I₂ flux |
| in glycerin (ppm) | Results (FIG. No.) | rate^{b} |
| 14,670 | No colony shown in the petri | 7.4 |
| | dish (FIG. 1A) | |
| 7,007 | No colony in vicinity of the I₂- | 3.5 |
| | infused catheter (FIG. 1B) | |
| 3,376 | FIG. 1C | 1.7 |
| 1,019 | FIG. 1D | 0.52 |
| 587 | FIG. 1E | 0.30 |
| 387 | FIG. 1F | 0.20 |
| 0 | FIG. 1G | 0 |
| N/A Blank | FIG. 1H | 0 |
| 738 | FIG. 2A | 0.39 |
| 301 | FIG. 2B | 0.087 |
| 136 | FIG. 2C | 0.029 |
| 25 | FIG. 2D | 0.0085 |
| 0 | FIG. 2E | 0 |
| N/A Blank | FIG. 2F | 0 |

^{a}Bardia Foley 100% Silicone Catheter, 30 cc, Size 24Fr (Lot# NGFS2195).
^{b}I₂ flux of I₂-infused catheter in μg I₂ per gram polymer per/second.

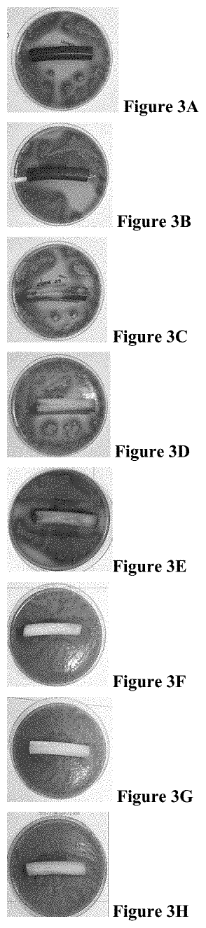

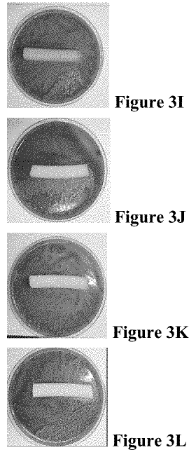

TABLE 2(B)-2
Biocidal effects of various embodiments of the molecular
iodine-infused latex catheters (Bard^{a} catheter tube material,
5 cm in length) on E. coli streaked agar plates.

| Molecular | | |
|---|---|---|
| iodine preparation | | |
| composition, I₂ conc. | | I₂ |
| in glycerin (ppm) | Results (FIG. No.) | flux^{b} |
| 12,560 | No colony in vicinity of the I₂- | 0.59 |
| | infused catheter (FIG. 3A) | |
| 10,100 | No colony in vicinity of the I₂- | 0.52 |
| | infused catheter (FIG. 3B) | |
| 7,961 | No colony in vicinity of the I₂- | 0.13 |
| | infused catheter (FIG. 3C) | |
| 5,831 | No colony in vicinity of the I₂- | 0.078 |
| | infused catheter (FIG. 3D) | |
| 4,161 | No colony in vicinity of the I₂- | 0.15 |
| | infused catheter (FIG. 3E) | |
| 3,688 | No colony near edges of the I₂- | 0.11 |
| | infused catheter (FIG. 3F) | |
| 3,305 | No colony near edges of the I₂- | 0.090 |
| | infused catheter (FIG. 3G) | |
| 3,196 | No colony near edges of the I₂- | 0.073 |
| | infused catheter (FIG. 3H) | |
| 2,858 | No colony near edges of the I₂- | 0.069 |
| | infused catheter (FIG. 3I) | |
| 2,025 | No colony near edges of the I₂- | 0.017 |
| | infused catheter (FIG. 3J) | |
| 998 | Colony near edges of the I₂-infused | 0.0039 |
| | catheter (FIG. 3K) | |
| 730 | Colony near edges of the I₂-infused | 0.0020 |
| | catheter (FIG. 3L) | |
| 373 | Colony near edges of the I₂-infused | 0 |
| | catheter (Figure not shown) | |
| 251 | Colony near edges of the I₂-infused | 0 |
| | catheter (Figure not shown) | |
| 100 | Colony near edges of the I₂-infused | 0 |
| | catheter (Figure not shown) | |

^{a}Bard latex Part 50615 (C. R. Bard, Inc., Covington, GA, Lot# NGFW1867).
^{b}I₂ flux of I₂-infused catheter material in μg I₂ per gram polymer per second.

TABLE 2(B)-3
Duration of biocidal iodine flux of various embodiments of
the molecular iodine-infused silicone catheters (Bardia^{b} or
Amsino^{c}) treated with molecular iodine preparation compositions
in different solvents (glycerin or propylene glycol).

| | | | | Duration of a | | |
|---|---|---|---|---|---|---|
| | | | | biocidal I₂ Flux | | Properties of |
| | | Molecular iodine | | I₂ flux of 0.19 | | the I₂-infused |
| | | preparation | | μg I₂ per second | | catheters |
| Catheter (pre- treatment) | | composition | | per gram of | | Initial I₂ |
| Catheter | Sample | | I₂ | polymer or | | concentration |
| model | size/shape | Solvent | (ppm) | higher^{b} | | (mg/g)^{a} |
| Bardia^{c} | 1 cm long | Glycerin | 19,400 | 10.4 | days | 13.0 |
| Amsino^{d} | intact tube | Glycerin | 19,400 | 24 | hours | 5.16 |
| Bardia^{c} | | Propylene | 19,400 | 10 | days | 4.54 |
| | | Glycol | | | | |
| Amsino^{d} | | Propylene | 19,400 | 5 | hours | 2.32 |
| | | Glycol | | | | |

^{a}I₂ concentration of I₂-infused catheter measured according to Example 3A.
^{b}I₂ flux in units of μg of I₂ per second per gram of polymer.
^{c}Bardia Foley 100% silicone catheter, 30 cc, size 24 Fr, C. R. Bard, Inc.
^{d}Amsure Foley 100% silicone catheter, 30 cc, size 24 Fr, Amsino International, Inc.

TABLE 2(B)-4
Biocidal effects^{a} of an embodiment of the molecular iodine-infused
catheters (Bardia^{b}, 5 cm) treated^{d} with molecular iodine
preparation compositions in glycerin (17,800 ppm)

| | | Molecular iodine | | | |
|---|---|---|---|---|---|
| | | preparation | | E. coli. | E. coli |
| | Catheter | composition | | dilution | dilution |
| | (pre-treatment) | Solvent | I₂ (ppm) | 1/500 | 1/1000 |
| I₂-infused | Bardia^{b} | Glycerin | 17,800 | No colony | No colony |
| catheter | | | | observed | observed |
| Glycerin | Bardia^{b} | Glycerin | None, | Colonies observed, similar to | |
| treated catheter | | | control | the respective blank control | |
| Catheter | Bardia^{b} | None, | None, | Colonies observed, similar to | |
| without | | control | control | the respective blank control | |
| treatment | | | | | |
| Blank control, | None | None | None | Colonies | Colonies |
| nothing added | | | | observed | observed |
| to the E. coli | | | | | |
| streaked agar | | | | | |
| plate | | | | | |

^{a}Each sample was placed on an agar plate (Diamante Scientific, DIAP1400; Lot# 93236) streaked with E. coli (10 μL, 1/500 or 1/1000 dilution of E. coli stock solution (0.1 mM phosphate buffer, pH 5.0, OD600 of 0.739)); the plates were imaged for bacterial growth monitor after 24 hours at 37° C.
^{b}Bardia Foley 100% silicone catheter, 30 cc, size 24 Fr, C. R. Bard, Inc.
^{c}treatment for 4 days, then rinsed with water and dried.

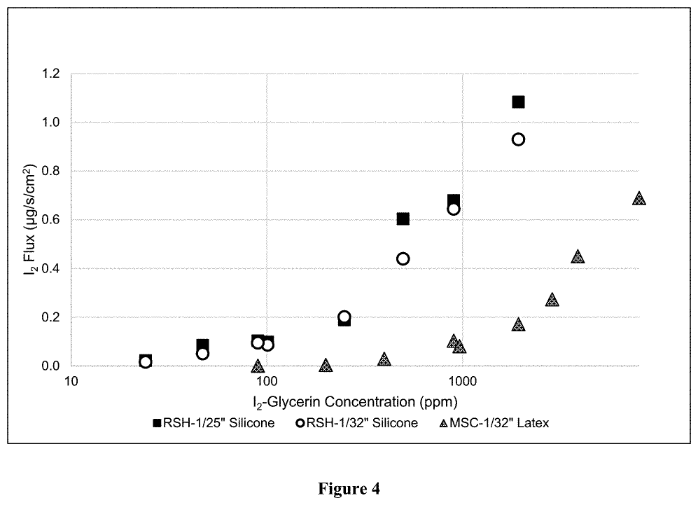

TABLE 2(B)-5
Biocidal effect^{a} of an embodiment of the molecular iodine-infused
polymers (silicone^{b&c} or latex,^{d} 1 cm × 1 cm) treated^{e} with
molecular iodine preparation compositions in glycerin with different
I₂ concentrations (47 to 7.984 PPM) (FIG. 4)

| Molecular iodine | | | |
|---|---|---|---|
| preparation composition | RSH- 1/25″^{b} | RSH- 1/32″^{c} | MSC- 1/32″^{d} |
| I₂ (ppm) | I₂ flux rate^{f} | I₂ flux rate^{f} | I₂ flux rate^{f} |
| 24 | 0.02 | 0.02 | — |
| 47 | 0.08 | 0.05 | — |
| 90 | 0.10 | 0.10 | 0.00 |
| 101 | 0.10 | 0.09 | — |
| 200 | — | — | 0.00 |
| 249 | 0.19 | 0.20 | — |
| 398 | — | — | 0.03 |
| 497 | 0.60 | 0.44 | — |
| 901 | 0.64 | 0.64 | 0.10 |
| 965 | — | — | 0.08 |
| 1,930 | 1.08 | 0.93 | 0.17 |
| 2,870 | — | — | 0.27 |
| 3,880 | — | — | 0.45 |
| 7,984 | — | — | 0.69 |

^{a}Each sample was placed on an agar plate (Diamante Scientific, DIAP1400; Lot# 94447) streaked with E. coli (10 μL, 1/1000 dilution of E. coli stock solution (0.1 mM phosphate buffer, pH 5.0, OD600 of 0.822)); the plates were imaged for bacterial growth monitor after 24 hours at 37° C.
^{b} 1/25″ thick Laimeisi rubber high temperature material, referred to as RSH- 1/25″.
^{c} 1/32″ thick Laimeisi rubber high temperature material, referred to as RSH- 1/32″.
^{d} 1/32″ thick latex sheets from MSC Industrial Company, referred to as MSC- 1/32″.
^{e}Treatment for 7 days, then rinsed with water and dried.
^{f}I₂ flux rate (μg I₂/second/cm²⁾ measured according to Example 3(B)(i), each dried sample was votexed in 5 mL DPD for 10 seconds, and the I₂ concentration of the obtained DPD solution was measured by absorbance at 550 nm.

Based on the observations in Table 2(B)-1 and Table 2(B)-2 an iodine flux of 0.20 μg I₂ per second per gram of polymer or higher was found to be biocidal for cylindrical pieces of Bardia and Amsino silicone catheters. The duration of a biocidal I₂ flux from I₂-infused silicone catheter material was evaluated after treatment with 19,400 ppm I₂-glycerin and 19,400 ppm I₂-propylene glycol. The duration of I₂ flux varied with both solvent and silicone material. The Bardia catheter exhibited a longer I₂ flux duration than the Amsino catheter. The duration of the I₂ flux from the Amsino catheter was longer when the catheter was treated with I₂-glycerin as compared to I₂-propylene glycol. The duration of biocidal I₂ flux is an important consideration because it is not practical to change an indwelling catheter daily for a patient who requires chronic usage of these devices. Consequently, the duration of a biocidal I₂ flux is an important characteristic of an I₂-infused catheter.

2(C). Biocidal Effects of I₂-Infused Silicone Material Prepared with I₂-Glycerin Preparation Composition or Aqueous Iodine Composition with Various Organisms.

Biocidal effects of silicone samples (1 cm×1 cm×0.08 cm, RSH-1/32) treated with molecular iodine preparation composition in glycerin (2,500 ppm) or aqueous solution of molecular iodine (200 ppm) were evaluated. The silicone samples were washed with sterile DI water three times, dried under sterile conditions and then used for biocidal evaluation on agar plates streaked with various organisms known to be responsible for urinary tract infections. Untreated silicone samples were used as negative controls. Blank controls were organism streaked agar plates without further treatment.

Test cultures were initiated from frozen glycerol stocks and streaked onto fresh plates of appropriate growth media. The obtained plates were incubated at 37±2 C and CO₂ overnight or until sufficient growth was observed. Single colony isolates were used to inoculate 5 mL of Tryptic Soy Broth (TSB) and the resulting suspensions were incubated with shaking at 37±2 C for 16-18 hours. 50 μL of the obtained suspensions were then inoculated with 5 mL of TSB and then incubated with shaking at 37±2 C for 3-6 hours or until sufficient turbidity was observed. The obtained subcultures were diluted to about 1.5×10E4 cfu/mL based on their OD600.

100 μL of inoculum was spread across plates of organism appropriate agar such that the total recoverable colonies after incubation would be between 300 to 1,500 cfu/plate. The silicone sample saturated with the molecular iodine preparation composition in glycerin (2,500 ppm) or the aqueous iodine composition (200 ppm) and the negative control (untreated) silicone material was respectively placed at the center of the agar plates streaked with an organism. All plates with a silicone material and blank control plates were incubated at 37±2 C and 5% CO₂ for 48 hours, and photographed at 24 and 48 hours, respectively. See Table 2C for summary.

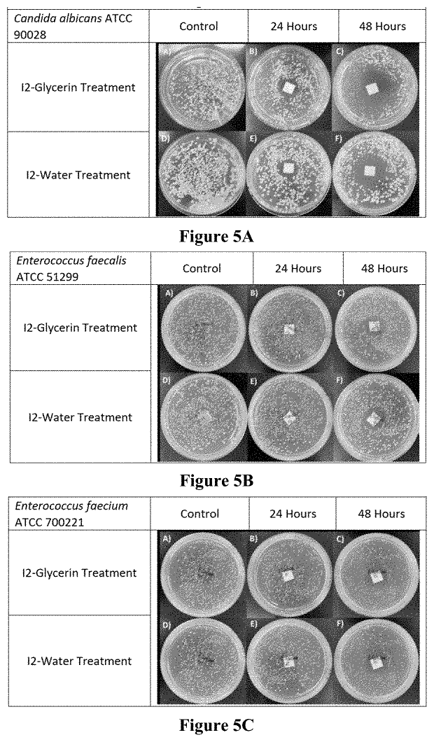

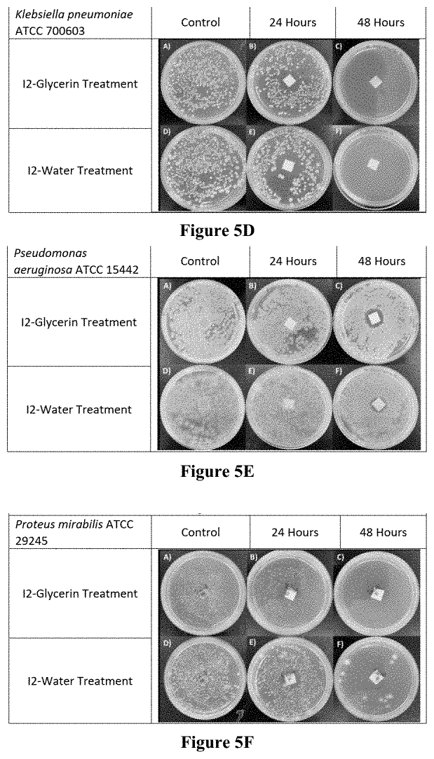

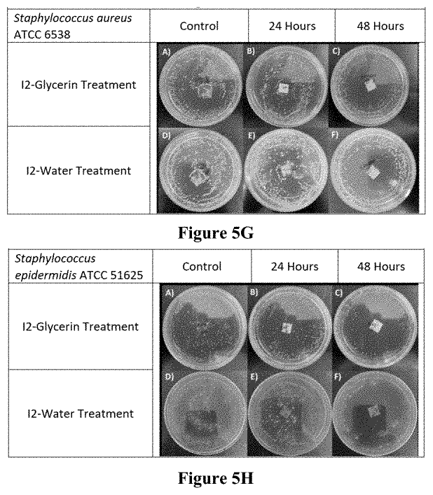

TABLE 2C
Biocidal effects of I₂-infused silicone material (1 cm × 1
cm) prepared with molecular iodine preparation composition in glycerin
(2,500 ppm) or a 60% saturated aqueous solution of I₂ (200 PPM)

| | Agar | | |
|---|---|---|---|
| Organism | media | FIG. | Biocidal Evaluation |
| C. albicans | SDA | FIG. 5A | The glycerin treated silicone demonstrated |
| ATCC ® | | | greater biocidal activity at 48 hours based on the |
| 90028 ™ | | | size of the zone of killing. |
| E. faecalis | BHI agar | FIG. 5B | Effective kill of this bacteria will require higher |
| ATCC ® | | | I₂ concentrations than what can be achieved with |
| 51299 ™ | | | water. |
| E. faecium | BHI agar | FIG. 5C | Higher concentrations of I₂ than used will be |
| ATCC ® | | | required to kill. This can be achieved with |
| 700221 ™ | | | glycerin but not in water which is required for |
| | | | efficacy. |
| K. pneumoniae | Nutrient | FIG. 5D | The low-I₂ glycerin treatment eliminated all |
| ATCC ® | agar | | colonies while 1 colony survived with the |
| 700603 ™ | | | aqueous treatment. |
| P. aeruginosa | TSA | FIG. 5E | I₂-glycerin was superior to the aqueous |
| ATCC ® | | | treatment. A higher concentration of I₂ is likely |
| 15442 ™ | | | required for clinical utility. |
| P. mirabilis | Nutrient | FIG. 5F | The I₂-glycerin treatment completely killed all |
| ATCC ® | agar | | P. mirabilis while the aqueous I₂ treatment did not. |
| 29245TM | | | |
| S. aureus | MSA | FIG. 5G | The I₂-glycerin treatment was superior to the |
| ATCC ® | | | aqueous treatment at both 24 and 48 hours. |
| 6538 ™ | | | |
| S. epidermidis | MSA | FIG. 5H | The I₂-glycerin treatment was superior to the |
| ATCC ® | | | aqueous treatment at 24 hours and more |
| 51625 ™ | | | effective at 48 hours. |

The maximum solubility of I₂ in water at room temperature is 332 ppm. Silicone samples treated with aqueous solution of molecular iodine (200 ppm) showed a quicker I₂ flux profile compared to silicone samples treated with molecular iodine preparation composition in glycerin.

Molecular iodine-infused samples treated with I₂-glycerin showed more effective biocidal effects than samples treated with aqueous molecular iodine solutions (FIGS. 5A to 5H). The concentration of Pseudomonas aeruginosa used was more representative of the bioburden a treated catheter would confront in a clinical infection and the larger zone of killing surrounding the I₂-glycerin treated silicone indicates that an aqueous treatment would be inferior for clinical applications.

2(D). Biocidal Effects of I₂-Infused Porous Chitosan Wound Dressing 2(D)(i). Biocidal Effect over E. coli

Embodiments of molecular iodine-infused articles were prepared by contacting a wound pad material (Sentrex BioSponge™ obtained from Binova, which was a porous chitosan wound dressing, 2 cm×2 cm) with 100 k ppm I₂ in propylene glycol, 10 k ppm I₂ in propylene glycol, 1 k ppm I₂ in propylene glycol, and 100 ppm I₂ in propylene glycol, respectively, for 30 minutes. The treated wound pad material was then patted with a paper towel to remove excess I₂ in propylene glycol.

E. coli was grown on McConkey media overnight and collected using a sterile loop after 24 hours. The bacterial was suspended in normal saline, diluted one hundred-fold and an optical density of 1.139 was observed at 600 nm. One hundred microliters of a 1/5,000 dilution of the E. coli bacterial suspension was spread across the surface of a plate of McConkey media and the bacteria were allowed to incubate at 37° C. for 5 hours.

The treated wound dressing samples were placed on top of the agar with actively growing E. coli overnight, and no bacteria grew underneath the treated wound dressing samples.

2(D)(ii). Biocidal Effect Over Pseudomonas aeruginosa or Staph aureus

The wound pad material (1 cm×1 cm) was treated with 120 μL I₂ in propylene glycol at concentrations of 500 ppm, 1 k ppm, 1.5 k ppm, 3 k ppm, 6 k ppm, and 9 k ppm, respectively, and then placed on top of agar with actively growing bacteria pseudomonas aeruginosa and staph aureus, respectively for 24 hours. Similarly, no bacteria grew underneath the treated material; however, several colonies of pseudomonas and Staph aureus growing adjacent to the material treated with 500 ppm I₂ in propylene glycol were observed.

### Example 3

### Molecular Iodine Concentration and I₂ Flux Rates of Embodiments of the Molecular Iodine-Infused Catheters and Materials

Biocidal effects of an embodiment of the molecular iodine-infused catheters were evaluated by 1) subjecting a sample of the embodiment of the molecular iodine-infused catheter to a microorganism challenge for a first period of time; and 2) evaluating the biocidal effects of the sample.

### 3(A). Molecular Iodine Concentration of Embodiments of the Molecular Iodine-Infused Catheters and Materials.

For catheters and materials (e.g., silicone and latex) that decompose in organic solvents such as toluene, the molecular iodine concentration of an embodiment of the molecular iodine-infused catheter or material was measured by 1) extraction from a piece of the embodiment of the molecular iodine-infused catheter tubing that is 1 cm in length or iodine-infused flat polymeric material 1 cm×1 cm×0.08 cm was submerged in 5 to 50 mL toluene to provide an iodine-toluene solution and decomposed catheter material (silicone, latex, etc . . . ); 2) measuring the molecular iodine concentration of the I₂-toluene solution by absorbance at 497 nm; and 3) calculating the molecular iodine concentration of the embodiment of the molecular iodine-infused catheter using a standard curve. See Table 3(A) for summary of molecular iodine concentrations of various embodiments of the molecular iodine-infused catheters and materials. Solvents other than toluene may be used to decompose an embodiment of the molecular iodine-infused catheter material, provided the molecular iodine or iodine species remain stable at least while measurements are made.

TABLE 3(A)
Molecular iodine concentrations of various embodiments
of the molecular iodine-infused silicone catheters
and materials saturated in I2-solvent (19,400 ppm).

| Catheter | | Iodine Concentration per Gram Polymer |
|---|---|---|
| Manufacturer | Solvent | (mg I₂/g Polymer) |
| Bardia¹ | Glycerin | 13.0 |
| Amsino³ | Glycerin | 5.16 |
| Bardia¹ | Propylene | 4.54 |
| | Glycol | |
| Amsino³ | Propylene | 2.32 |
| | Glycol | |

¹Bardia Foley 100% silicone catheter, 30 cc, size 24 Fr (C. R. Bard, Inc., Ref# 806324; Lot# NGFS2346).
³Amsure Foley 100% silicone catheter, 30 cc, size 24 Fr (Amsino International, Inc., Ref# AS42024S, Lot#160017WL).

### 3(B) Measurement of Iodine Flux Rates of Embodiments of the Molecular Iodine-Infused Catheters and Materials.

Iodine flux rates of an embodiment of the molecular iodine-infused catheter were measured in Examples 3(B)(i) through 3(B)(iii) below. Unless specified otherwise, iodine flux rates referred to in the Example section were measured according to the method disclosed in Example 3(B)(i).

### 3(B)(i) Measurement of Iodine Flux of a 5-cm Sample in 100 mL DPD

An aqueous solution of 1 mM N,N-dimethyl-p-phenylenediamine dihydrochloride (DPD) was stirred at 250 rpm with a 50 mm PTFE-lined stir bar in a glass beaker. A sample of an I₂-infused infused polymer (in this case a 5 cm piece of catheter) that had been washed with water and dried was placed in the beaker of DPD under stirring. The I₂-infused material was stirred in the DPD solution for a time period that was sufficient to allow color to be formed and then a sample was removed to measure the absorbance (λ=550 nm); the absorbance intensity was converted into I₂ concentration using the known extinction coefficient for the chromophore formed between I₂ and DPD.

### 3(B)(ii) Measurement of Iodine Flux of a 1-cm Sample in DPD

The method described in Example 3(B)(i) was used.

See Table 3(B)(ii) for summary of iodine flux rates of various embodiments of the molecular iodine-infused catheter measured as described in instant Example 3(B)(ii).

The I₂ flux of the I₂-infused Bardia Foley silicone catheter (Sample A) decreased by more than 65% over the first 15 minutes from an initial level of 2.09 μg I₂ per gram polymer per second. The I₂ flux of the I₂-infused Hollister latex catheter (Sample E) also decreased over the first 15 minutes from an initial level of 1.21 μg I₂ per gram polymer per second.

TABLE 3(B)(ii)
Iodine flux rates of various embodiments of the molecular iodine-infused
catheters and various catheter treated with aqueous I₂ solutions.

| | | | | | | | | Time when |
|---|---|---|---|---|---|---|---|---|
| I₂ Outgassed | The Catheter | | Molecular iodine | | | Infusion | I₂ flux at | the I₂ |
| between | (pre- treatment) | | preparation composition | | I₂ flux | time | min. 2-4 | flux rate |
| Minute 2 to 4 | Catheter | Catheter | | Molecular I2 | rates | (hrs) | (μg I₂/cm² | dropped 90% |
| (ug/min/gram) | Manufacturer | material | Solvent | (ppm) | (FIG.) | — | sample) | (min) |
| A | Bardia¹ | Silicone | PG | 17k | FIG. 7 | — | — | — |
| E | Hollister⁶ | latex | PG | 17k | FIG. 7 | — | — | — |
| | Bardia¹ | Silicone | Aq | 10% PVP-I | — | 23 | 0 | N/A |
| | | | | (2-6 ppm I₂) | | | | |
| | Bard² | latex | Aq | 10% PVP-I | — | 23 | 00013 | 18 |
| | | | | (2-6 ppm I₂) | | | | |
| | Bard² | latex | EtOH | 10k | — | 19.8 | — | 48 |
| | Bard² | latex | EtOH | 100k | — | 19 | 0.0088 | 27.5 |
| | John Guest⁷ | LLDPE | EtOH | 10k | — | 21.5 | 0.00054 | 10 |
| | John Guest⁷ | LLDPE | EtOH | 100k | — | 18 | 0..0031 | 76 |

¹Bardia Foley 100% silicone catheter, 30 cc, size 24 Fr (Ref# 806324, Lot# NGFS2195).
²Bard latex Part 50615 (C. R. Bard, Inc., Covington, GA, Lot# NGFW1867).
⁶Hollister latex male external condom catheter (Ref# 9107, Lot# 1C078A),
⁷John Guest linear low-density polyethylene (LLDPE, Middlesex, UK, Ref# PE-08-BI-0500F-N).

3(B) (iii) Measurement of I₂ Flux from I₂-Infused Catheter in Ambient Environment

The I₂ flux for 1×1 cm samples of embodiments of molecular iodine-infused catheter material was determined after the sample was placed in an ambient environment (20° C., Ex. 3(B)(iii)-1), or at 37° C. (Ex. 3(B)(iii)-2) for a desired time period (e.g., 0, 0.5, 1, 2, 3, 4, 5, 24, 48, 72, 96, 120, 240, 480, and 720 hours, or longer or other time periods). At each measurement time point, the I₂-flux was measured as described in Example 3(B)(i). See Table 3(B)(iii) for summary of iodine flux rates of various embodiments of the molecular I₂-infused catheter measure according to Ex. 3(B)(i). The duration of I₂ outgassing with all the iodine treatments identified in Table 3(B)(iii) were much less than that observed with glycerin. The iodine flux rates were measured up to 24 hours as the flux rates reduced below the iodine flux sufficient to kill the test bacteria about or before 24 hours. The minimum biocidal iodine flux was reached in 15 hours with Lugol's solution; in 24 hours with Iodine solution; in 24 hours with Iodine Tincture; in 0 hours with Povidone; in 8 hours with aqueous I_{2;} and in 6 hours with aqueous I₂ with methanol.

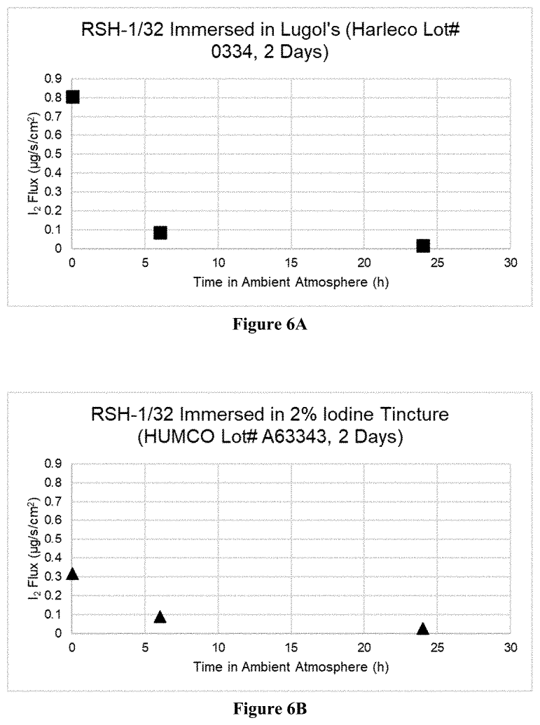

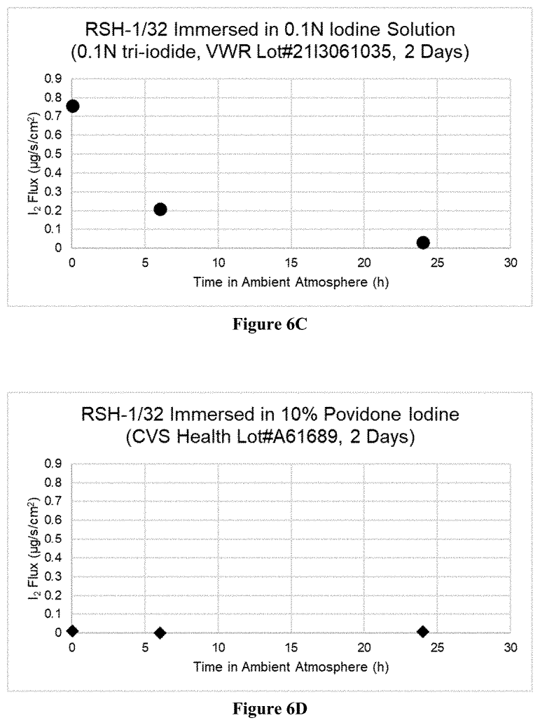

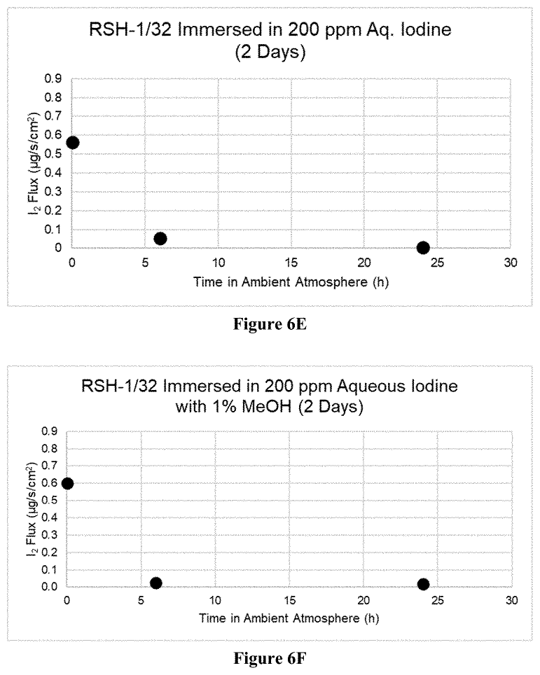

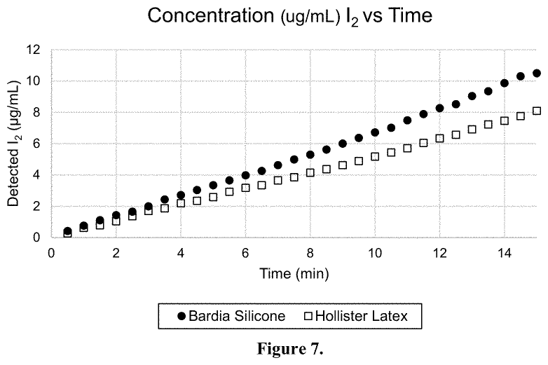

TABLE 3(B)(iii)
I₂ flux rates of I₂-Infused flat silicone
saturated in various aqueous I₂ solutions.

| | Titratable Iodine | I₂ flux¹ of the |
|---|---|---|
| | Concentration (ppm, Lab | I₂-infused |
| Solvent | Labeled/Measured) | silicone material |
| Lugol's Iodine Solution | 3,330/2,750 | FIG. 6A |
| Iodine Tincture | 20,000/17,300 | FIG. 6B |
| Iodine Solution | 1,000-10,000/10,900 | FIG. 6C |
| Povidone-I₂ solution | 10,000/8400 | FIG. 6D |
| Aqueous I₂ | —/200 | FIG. 6E |
| Aqueous/I₂ Solubilized | —/200 | FIG. 6F |
| with Methanol (~1% by | | |
| wt) | | |

3(B)(iv) I₂ Flux Rates Measured may be Affected by the Length and Shape of the I₂-Infused Catheters.

I₂-infused silicone catheter materials with different lengths or different shapes were removed from the molecular iodine preparation composition, washed with water, dried on a paper towel and then the I₂ flux was immediately measured, see Table 3(B)(iv). Samples that were halved longitudinally showed higher I₂ flux rates while the impact of different lengths varied. Nevertheless, the measurements made were reproducible.

TABLE 3(B)(iv)
Impact of length and shape of I₂-in fused catheters on I₂ flux rates
Preparation composition: I₂-glycerin (14,000 ppm)

| | I₂ flux rate (μg/gram polymer/second) | | | | I₂ flux rate (μg/cm² polymer/second) | | | |
|---|---|---|---|---|---|---|---|---|
| | Length | | | | | | | |
| | 1 cm | | 5 cm | | 1 cm | | 5 cm | |
| | Shape | | | | | | | |
| | Intact | Halved | Intact | Halved | Intact | Halved | Intact | Halved |
| | tubing | longitudinally | tubing | longitudinally | tubing | longitudinally | tubing | longitudinally |
| Bardia¹ | 5.8 | 7.2 | 7.1 | 9.4 | 0.39 | 0.63 | 0.39 | 0.74 |
| Amsino³ | 4.9 | 5.0 | 5.5 | 6.3 | 0.28 | 0.34 | 0.41 | 0.49 |

¹Bardia Foley 100% silicone catheter, 30 cc, size 24 Fr, C. R. Bard, Inc., Ref# 806324;
³Amsure 100% silicone foley catheter, 30 cc, size 24 Fr (Amsino International, Inc., Ref# AS42024S; Lot# 160017WL)

3(B)(v) I₂ Flux of I₂-Infused Silicone Treated with Molecular Iodine Preparation Compositions with Different Solvents (Glycerin and Propylene Glycol (PG))

Silicone treated with molecular iodine preparation compositions with different solvents (glycerin and propylene glycol (PG)) but the same I₂ concentrations showed different I₂ flux rates. I₂ infusion of two silicone catheters [Bardia Foley 100% Silicone Catheter, C. R. Bard, Inc., Covington, Ga., 30 cc, Size 24 Fr (Ref#806324, Lot#NGF S2195) and Amsino AMSURE 100% Silicone Foley Catheter, International, Inc., Pomona, Calif., 30cc, Size 24Fr (Ref# AS420245; Lot#152018WL) were compared using I₂ concentrations of 17,150 ppm in either propylene glycol or glycerin. Catheter materials of 1 cm in length were placed in 15 mL of molecular iodine preparation composition contained in 20 mL scintillation vials (Qorpak GLC-00999) and allowed to rotate (Roto-Shake Genie Rotator, Scientific Industries Genie SI-1100 Roto-Shake Rotator/Rocker; 120 VAC; MFR#SI-1100) on speed setting 2 for 4 days. The Amsure catheter material saturated in I₂-glycerin yielded an iodine flux rate of 271 μg/g polymer/s. Under identical treatment conditions the Bardia catheter material yielded an iodine flux rate of 315 μg/g polymer/s. The Amsure catheter material saturated in I₂-propylene glycol yielded an iodine flux rate of 26 μg/g polymer/s. Under identical treatment conditions the Bardia catheter material yielded an iodine flux rate of 43 μg/g polymer/s.

TABLE 3(B)(v)
I₂ flux rates of I₂-Infused silicone treated with molecular iodine
preparation compositions with different solvents (glycerin or PG)

Molecular iodine preparation compositions

| | I₂ concentration | Initial I₂ flux rates |
|---|---|---|
| Solvent | (PPM) | (μg I₂/g/sec) |
| Glycerin | 16k | 5.7 |
| PG | 72k | 1.81 |

3(B) (vi) I₂ Release of I₂-Infused Silicone Treated with Molecular Iodine Preparation Compositions in Propylene Glycol (PG)

Embodiments of molecular iodine-infused articles were prepared by contacting a Bardia silicone catheter and a Hollister latex catheter with high molecular iodine concentration composition (17,000 ppm I₂ in propylene glycol) for 10 days on a Roto-Shake Genie at a setting of 2 (Scientific Industries). A sample was taken from each molecular iodine-infused catheters (0.305 g Bardia silicone and 0.435 g Hollister latex catheter), washed with water, placed in distilled water and patted dry. The iodine flux was determined as described in Example 3(B)(i). Data showed approximately linear I₂ outgassing from both catheter samples at least for the first 200 s (FIG. 7).

### Example 4

### Visual Observations of I₂ Flux from Various Embodiments of the Molecular Iodine-Infused Catheters and Materials

Embodiments of molecular iodine-infused catheters and polymers were prepared as specified in Table 4A and I₂ flux rates of the embodiments were measured according to the method used in Example 3B(ii).

Pictures of color change in molecular iodine-infused catheters, articles, and materials were taken with a Moultrie Wingscapes TimelapseCam Pro (Model# WCT-00126) at room temperature for various times (e.g., up to 43 days for FIGS. 8A-8G, as summarized in Table 4). For example, FIG. 8A shows the region-specific sequestration of I₂ and subsequent release (outgassing) of I₂ from a sample of a silicone breast implant. Timepoints are shown at 1, 3, 4, 8, 24,32, 48, 51 and 56 hours. The images demonstrate that regions of the silicone implant sequester and release I₂ with different avidity.

TABLE 4
Uptake and release of I₂ of embodiments of the
molecular iodine-infused polymer or articles

| | | Molecular iodine | | | |
|---|---|---|---|---|---|
| | | preparation composition | | Visual | |
| Catheter/ | | | I₂ Conc. (ppm)/ | observation | |
| Material | Polymer | Solvent | treatment time | FIG. No. | Comments |
| Natrelle Breast | Silicone | Glycerin | 17,000/90 hrs | FIG. 8A | Oscillatory |
| Implant¹ | | | | | behavior of I₂ |
| | | | | | capture/release |
| | | | | | in silicone, |
| | | | | | color faded |
| | | | | | away, turned |
| | | | | | dark again |
| | | | | | (32 hr), |
| | | | | | faded, turned |
| | | | | | darker again |
| | | | | | (51 hr) and |
| | | | | | faded |
| Bardia², | Silicone | Propylene | 5k, 10k, 20k, | FIGS. | Time 0, 37.5 |
| Amsure³, | and latex | glycol | 40k, or 80k | 8B-8D | and 208 |
| Cold&Coler⁴, | | | | | hours post |
| Nipple Covers⁵ | | | | | treatment |
| and Hollister | | | | | |
| latex⁶ | | | | | |
| Bardia² | Silicone | Glycerin | 10,000 | FIG. 8E | |
| Bardia² | Silicone | Propylene | 10,000 | FIG. 8F | |
| | | glycol | | | |
| Amsure⁴ | Silicone | Propylene | 10,000 | FIG. 8G | |
| | | glycol | | | |

¹Natrelle Inspira, 340 cc; style SRX; Lot 3089145 from Allergan, Irvine CA
²Bardia Foley 100% Silicone Catheter, 30 cc, Size 24 Fr (Ref# 806324, Lot# NGFS2195).
³Amsure 100% silicone foley catheter, 30 cc, size 24 Fr, (Amsino International, Inc., Ref# AS42024S; Lot# 160017WL).
⁴Cold and Colder, Ultra Clear Platinum Silicone Tubing - Food & Medical Grade - High Temp Hose, ½″ ID × ⅝″ OD.
⁵FJYQOP Silicone Nipple Covers.
⁶Hollister latex male external condom catheter (Ref# 9107, Lot# 1C078A).

### Example 5

### I₂-Infused Catheters and Materials Prepared with Molecular Iodine Preparation Compositions in Various Solvents

Various polymer or article samples were treated with different molecular iodine preparation compositions as summarized in Table 5A. Some samples were not stable physically or chemically in certain solvents after a long period of exposure. The rate of I₂-infusion into the polymers were different for different materials treated with the same molecular iodine preparation composition. I₂-loading rates were different for the same materials treated with different molecular iodine preparation compositions (e.g., different solvent, different I₂ concentration). Very limited or no amount of I₂ was infused into PET and PTFE materials after immersion in I₂-ethanol containing 100,000 ppm I₂ after 3 days and 5 days, respectively. I₂-ethanol provided faster I₂ loading for multiple silicone samples (reached I₂ saturation by day 2) than molecular iodine preparation compositions with solvents such as propylene glycol, glycerin, dimethyl sulfoxide, and isopropanol, which required 3-4 days for I₂ saturation. However, silicone exposed to I₂-ethanol in this example decomposed after one day and the same silicone materials decomposed after two days in I₂-isopropanol. Surprisingly, ethanol alone did not alter the physical properties of silicone catheters in the absence of I_{2.} However, physical degradation of silicone was observed in an I₂ composition in a mixture solvent that included 20% or higher ethanol. Latex samples tested in this example were saturated with I₂ by day 2 in I₂-ethanol or in I₂-isopropanol, as well as in I₂-propylene glycol which had only half of the I₂ concentration of both I₂-ethanol and I₂-isopropanol.

TABLE 5A
I₂-infused materials with molecular iodine preparation
compositions in different solvents.

| | | I₂ | |
|---|---|---|---|
| | | Concentration | |
| Material | Solvent | (ppm) | Observation |
| Silicone | Propylene | 54,000 | Saturated by day 4 |
| | Glycol | | |
| Silicone | DMSO | 100,000 | Saturated by day 3 |
| Silicone | Glycerin | 24,000 | Saturated by day 4 |
| Silicone | Ethanol | 100,000 | Physically affected after day 1 |
| Silicone | Isopropanol | 100,000 | Saturated by day 2, polymer was |
| | | | physically affected by day 2 |
| Latex | Propylene | 54,000 | Saturated by day 2 |
| | Glycol | | |
| Latex | DMSO | 100,000 | Polymer decomposed by day 3 |
| Latex | Glycerin | 24,000 | Saturated by day 3 |
| Latex | Ethanol | 100,000 | Saturated by day 2 |
| Latex | Isopropanol | 100,000 | Saturated by day 2 |
| PTFE | Ethanol | 100,000 | Very limited or no I₂ uptake |
| LLDPE | Ethanol | 100,000 | Saturated by day 2 |
| PET | Ethanol | 100,000 | Very limited or no I₂ uptake |

### Example 6

### Methods to Prepare Pretreatment Molecular Iodine Composition

### A. Preparation of an I₂-Glycerin Composition According to an Embodiment of the Disclosure

A total of 33.74 grams of analytical grade elemental iodine (United Chemicals, Wuxi, Jiangsu, China Lot# 2020-01-01) was placed in a 240 mL glass borosilicate jar that had a PTFE screw top. A total of 144.24 grams of glycerin (Spectrum Chemicals, New Brunswick, N.J. 08901, Lot# 21E0215) was weighed into the glass jar on top of the elemental iodine. A Circulus™ magnetic stir bar (VWR, Radnor, Pa. 19087, Cat# 58947-849) was used to vigorously disperse the elemental iodine with the glycerin at a spin rate of 300 rpm during the initial 23 days, 150 rpm for the next 30 days and then without agitation for the remaining time. At different time points the mixing was stopped and less than 1 mL of material was removed to measure the absorbance at 460 nm in a Cole Parmer 1100 Spectrophotometer. Even under vigorous agitation it took a long time for the elemental iodine to dissolve in the glycerin such that it was impractical to contemplate preparing batches of material for commercial sales.

### B. Another Preparation of an I₂-Glycerin Composition According to an Embodiment of the Disclosure

An alternate approach was undertaken to prepare a dispersion of elemental iodine of glycerin. Prior experiments had demonstrated that iodine was not stable in ethanol as there was a loss of over 15% of molecular iodine after 15 days at room temperature when stored at room temperature in the dark. A total of 15.15 grams of molecular iodine was dissolved in 80 mL of absolute ethanol (ThermoFisher Scientific, Fair Lawn, N.J., 07410, Lot#B0538618A) and the volume was brought to a total of 100 mL ethanol resulting in an ethanol-iodine solution having 150,000 ppm molecular iodine. A five-liter chamber was charged with 4 liters of glycerin and the glycerin was stirred using a rotary mixer. Once the glycerin reached steady state of agitation the iodine-ethanol mixture was added dropwise to the glycerin over a period of 20 minutes. The mixture was continuously mixed and every five minutes a 0.5 mL volume was removed and an absorbance reading at 460 nm was taken. The mixture was homogenous within 45 minutes as judged by identical values of three successive absorbance readings which indicated a total molecular iodine concentration of 1,500 ppm (wt/v). The iodine species of the I₂-glycerin composition prepared was substantially molecular iodine. The controlled addition of a concentrated ethanol-iodine solution into an organic carrier provides the ability to accurately prepare compositions that contain high concentration of molecular iodine. The stability of molecular iodine in this composition did not exhibit any loss at room temperature over the first monitoring period which was 113 days total and over the second monitoring period which was 480 days total.

### C. Another Preparation of an I₂-Glycerin Composition According to an Embodiment of the Disclosure

Analytical grade molecular iodine (United Chemicals, Wuxi, Jiangsu, China Lot# 2020-01-01) was dissolved in propylene glycol (Alfa Asear, Lot 10225033) to a concentration of 10 grams per 100 mL or 100,000 ppm (w/v). This solution was used as an iodine concentrate and diluted to a final concentration of 1,500 ppm of molecular iodine in the following compositions: pure glycerin, pure propylene glycol, propylene glycol with 10% citric acid, propylene glycol with 5% citric acid, and propylene glycol with 1% citric acid.

1. A molecular iodine-infused catheter comprising a catheter and molecular iodine, the molecular iodine-infused catheter releasing an iodine flux.

2. The molecular iodine-infused catheter of claim 1 having one or more biocidal effects selected from the group consisting of biostatic persistent, biocidal persistent, and prolonged biocidal activities.

3. The molecular iodine-infused catheter of any one of the preceding claims, having a molecular iodine concentration of at least about 2.32 mg I_{2/}g or at least about 13.0 mg I_{2/}g.

4. The molecular iodine-infused catheter of any one of the preceding claims, having an iodine flux rate of at least about 0.19 μg I_{2/}sec/g.

5. The molecular iodine-infused-infused catheter of any one of the preceding claims, having biostatic persistent activity for at least 5 hours or for at least 10 days.

6. The molecular iodine-infused-infused catheter of any one of the preceding claims, having at least a 1 log, 2 log, 3 log, 4 log, 5 log, or 6 log biocidal persistent activity.

7. The molecular iodine-infused-infused catheter of any one of the preceding claims, the catheter comprising silicone or latex.

8. A method of treating or preventing UTI in a subject comprising contacting the molecular iodine-infused catheter of any one of the preceding claims with the subject.

9. The method of claim 8, wherein the molecular iodine-infused catheter of claim 1 or 2 remains in the subject for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, or 3 months.

10. The method of claim 8 or 9, further comprising delivering a molecular iodine in-use composition via the molecular iodine-infused catheter.

11. A molecular iodine-infused polymer comprising a polymer and molecular iodine, having a molecular iodine concentration of at least 2.32 mg I_{2/}g polymer or at least 13.0 mg I_{2/}g polymer.

12. A molecular iodine-infused polymer comprising a polymer and molecular iodine, emitting an iodine flux of at least about 0.19 μg I_{2/}sec/g polymer.

13. The molecular iodine-infused polymer of claim 12, emitting an iodine flux of at least 0.19 μg I_{2/}sec/g polymer for at least about 5 hours or for at least about 10 days.

14. The molecular iodine-infused-infused polymer of any one of claims 11-13, having biostatic persistent activity for at least 5 hours, or at least 10 days.

15. The molecular iodine-infused-infused polymer of any one of claims 11-14, having at least a 1 log, 2 log, 3 log, 4 log, 5 log, or 6 log biocidal persistent activity.

16. The molecular iodine-infused polymer of any one of claims 11-15, the polymer is selected from the group consisting of silicone, latex, chitosan, and combinations thereof.

17. A molecular iodine-infused article comprising the molecular iodine-infused polymer of any one of claims 11-16, the article is in a form selected from the group consisting of catheter, implant, wound dressing material, artificial skin, and bandage.

18. A method for treating or preventing a condition in a subject comprising contacting the subject with the molecular iodine-infused polymer of any one of claims 11-16 or the molecular iodine-infused article of claim 17, the condition being related to microorganism.

19. The method of claim 18, further comprising keeping the molecular iodine-infused polymer of any one of claims 11-16 or the molecular iodine-infused article of claim 17 in the subject for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, or 3 months.

20. The method of claim 18 or 19, the conditions treatable or preventable selected from the group consisting of urinary tract infection, breast implant infections, and wound infections.

21. The method of claim 18 or 19, wherein the molecular iodine-infused articles are wound dressing materials or artificial skin.

22. The method of claim 18 or 19, wherein the molecular iodine-infused article is a breast implant.
